(19) United States
(12) Patent Application Publication
(10) Pub. No.: US 2002/0188103 A1
(43) Pub. Date: Dec. 12, 2002

(54) CHIMERIC DNA-BINDING/DNA METHYLTRANSFERASE NUCLEIC ACID AND POLYPEPTIDE AND USES THEREOF

(76) Inventor: TIMOTHY H. BESTOR, NEW YORK, NY (US)

Correspondence Address: JOHN P WHITE, COOPER & DUNHAM, 1185 AVENUE OF THE AMERICAS, NEW YORK, NY, 10036

(21) Appl. No.: 09/051,013

(22) PCT Filed: Sep. 27, 1996
(86) PCT No.: PCT/US96/15576

(51) Int. Cl. C07K 1/00

(52) U.S. Cl. 530/350; 435/320.1; 435/325; 435/455; 435/456; 435/458; 435/459; 435/461; 424/93.2; 514/44; 536/23.1; 536/23.2; 536/23.5; 800/13

(57) ABSTRACT

The present invention provides a chimeric protein which comprises a mutated DNA methyltransferase portion and a DNA binding protein portion that binds sufficiently close to a promoter sequence of a target gene which promoter sequence contains a methylation site, to specifically methylate the site and inhibit activity of the promoter and thus inhibit expression of the target gene. This invention also provides for a method for inhibiting the expression of a target gene which includes contacting a promoter of the target gene with the chimeric protein, so as to specifically methylate the promoter sequence of the target gene thus inhibiting expression of the target gene.

This application claims the benefit of U.S. Provisional Application No. 60/004,445, filed Sep, 28, 1995, and this application is a continuation-in-part of U.S. Ser. No. 08/594,866, filed Jan. 31, 1996, the contents of which are hereby incorporated by reference into the present application.

### BACKGROUND OF THE INVENTION

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

All mammalian promoters tested to date have been found to be silenced when they contain 5-methylcytosine (m⁵C) at CpG sites (reviewed by Bestor, 1990; Meehan et al., 1993). Methylation represses transcription directly by interference with binding of transcription factors (Joel et al., 1993, and references therein) and methylated sequences are assembled into condensed chromatin that is inaccessible to transcription factors (reviewed by Bird, 1992). It has been shown that methylation of CpG dinucleotides in the 5′ long terminal repeat (LTR) of HIV-1 suppresses viral transcription (reviewed by Bednarik, 1992). Methylation can reduce transcription from the HIV-1 5′ LTR to undetectable levels (Joel et al., 1993). It is also known that methylation patterns are transmitted by clonal inheritance (Wigler, 1981), and that this is due to the strong preference of mammalian DNA methyltransferase for hemimethylated DNA (Bestor and Ingram, 1983; Bestor, 1992). The heritability of methylation patterns causes the affected promoters to be irreversibly inactivated, as has been observed for a large number of genes in cultured cells (reviewed by Holliday, 1993) and in human tissues (Yoshiura et al., 1995; Herman et al., 1994). While the exact function of DNA methylation remains the subject of discussion, its ability to suppress transcription is not in doubt.

Most exogenous nucleic acid sequences that a resident in a cell such as transposable elements and proviral DNA are methylated and inactivated in the genomes of mammals, flowering plants, and those fungi whose genomes contain m⁵C. Retroviral DNA is especially prone to inactivation by de novo methylation upon germline transmission, and this has greatly reduced the usefulness of retroviral transducing vectors in the construction of transgenic animals (Jähner and Jaenisch, 1984; Jaenisch et al., 1985). Treatment of cultured cells or mice with the demethylating drug 5-azacytidine (an irreversible inhibitor of DNA methyltransferases) can reactivate methylated retrovirus genomes (Jaenisch et al., 1985), and endogenous genes that have been silenced by ectopic de novo methylation of promoter regions can also be reactivated by demethylation (Holliday, 1993; Yoshiura et al., 1995). It is notable that organisms whose DNA lacks m⁵C (such as Drosophila) suffer far larger numbers of insertion mutations (Ashburner, 1992), which may reflect a reduced capacity for the control of mobile elements. These observations, together with evolutionary considerations (Bestor, 1990), strongly suggest that cytosine methylation is part of a genomic host defense system that limits the proliferation of parasitic sequence elements (Bestor, 1990; Bestor and Coxon, 1993). The selective advantage of such a defensive system is obvious, given that a significant fraction of the genome represents exogenous sequences that are invisible to the immune system and which might impose a lethal mutagenic or cytotoxic burden if allowed to proliferate unchecked.

The proviral DNA of many retroviruses is propagated in the repressed, latent state as a result of methylation of LTR sequences (reviewed by Jahner and Jaenisch, 1984). A body of evidence indicates methylation can cause latency in cells infected with HTLV-1 (Cassens and Ullrich, 1993) or HIV-1 (Bednarik et al., 1990; Joel et al., 1993 reviewed by Bednarik, 1992). The importance of methylation in latent HIV-1 infections in patients is unknown, but as HIV has only recently entered human populations, the host-parasite relationship may be far from equilibrium (Baltimore, 1995) and HIV proviral DNA may be inactivated with low efficiency under normal conditions (McCune, 1995). It is clear, however,. that HIV-1 transcription is very sensitive to methylation of 5′ LTR sequences (reviewed by Bednarik, 1992).

### SUMMARY OF THE INVENTION

The present invention provides a chimeric protein which comprises a mutated DNA methyltransferase portion and a DNA binding protein portion that binds sufficiently close to a promoter sequence of a target gene, which promoter sequence contains a methylation site, to specifically methylate the site and inhibit activity of the promoter and thus inhibit expression of the target gene. This invention also provides for a method for inhibiting the expression of a target gene which includes contacting a promoter of the target gene with the chimeric protein, so as to specifically methylate the promoter sequence of the target gene thus inhibiting expression of the target gene.

### BRIEF DESCRIPTION OF THE FIGURES

FIG. 1. Distribution of CpG sites and transcription factor binding sites in the 5′ LTR of HIV-1 (Sequence I.D. No. 1). CpG sites that fall within the recognition sequence of a restriction methyltransferase or methylation-sensitive endonuclease are shown above downward-pointing arrows. Data are from Garcia and Gaynor, 1994.

FIG. 2. Sequence of HIV-1 5′ LTR. CpG dinucleotides are bold and underlined; CpG sites that lie within the recognition sequence of methylation-sensitive restriction endonuclease are bold and italicized. Major transcription start site is denoted by +1, and the underlined sequence following denotes the transcribed sequence; numbering is with respect to the transcription start site. Please refer to FIG. 1 for location of protein binding sites. Strike through indicates the 9 bp binding site for the 3 finger protein developed by Wu et al. (1995). Sequence and numbering from Garcia and Gaynor, 1994.

FIG. 3. Gene silencing via targeted DNA methylation. A chimera between a sequence-specific DNA binding protein and a DNA methyltransferase with attenuated DNA binding directs methylation only to sites in the vicinity of the recognition-sequence of the DNA binding protein.

FIG. 4. Domain organization of mammalian DNA methyl-transferase. This enzyme is comprised of a long N-terminal regulatory domain and a C-terminal catalytic domain that closely resembles bacterial restriction methyltransferase. See Bestor and Verdine (1994) for a recent review.

FIG. 5. Protein-DNA contacts in the M. HaeIII-DNA transition state intermediate. The target cytosine is everted from the helix during methyl transfer, and is shown in the extra helical position. Thin dotted lines indicate base-specific contacts in the major groove, bold lines indicate contacts with the sugar-phosphate backbone. Mutation of residues involved in the sugar-phosphate contacts will reduce the affinity of DNA methyl-transferases for DNA, as will framework mutations that alter the disposition of the contact residues. Such mutations will make methyl transfer dependent on the DNA binding protein moiety of a DNA binding protein/DNA methyltransferase chimera, as described herein. Data from Reinisch et al. (1995).

FIG. 6. pLS, an expression construct to be used in the selection of DNA binding protein/DNA methyltransferase chimeras that methylate predetermined sequences. Lex A was chosen for its ability to direct fused proteins to Lex A binding sites on DNA (Brent and Ptashne, 1986), and M.SssI was chosen because of its high specific activity and the fact that it is the only bacterial methyltransferase that has the same specificity (5′-CpG-3′) (Renbaum and Razin, 1992) as the mammalian enzyme (Bestor and Ingram, 1983).

FIG. 7. Cyclic in vitro/in vivo selection for DNA binding protein/DNA methyltransferase chimeras in which methylation is dependent on the DNA binding protein moiety. McrBC encodes a nuclease that degrades heavily methylated DNA. Unmethylated DNA that has been linearized by a methylation-sensitive endonuclease does not transform. Use of these selective procedures on combinatorial libraries allows selection of chimeric DNA binding protein-DNA methyltransferase that target methylation only to a unique target site. This selection scheme may be applied to LexA/DNA methyltransferase chimeras (Example 1) and to zinc finger/DNA methyltransferase chimeras (Examples 3-5).

FIG. 8. Identification of CpG sites in the HIV-1 5′ LTR that yield maximal suppression of transcription when methylated. Individual 20mer primers that contain single m⁵CpG sites are hybridized to single-stranded M13 clones that contain the HIV-1 LTR and the bacterial CAT reporter gene. Extension of the primers with a DNA polymerase (sequenase) yields double-stranded DNA that is methylated on one strand at one CpG site. Upon transfection the hemimethylated site rapidly becomes methylated (Busslinger et al., 1983). The positions of each of the 11 CpG sites in the HIV-1 5′ LTR are given with respect to the transcription start site in the HIV-1 genome (Garcia and Gaynor, 1993).

FIG. 9. Phage-display selection of a zinc finger proteins that bind to predetermined sequences in the HIV-1 5′ LTR. The strategy used to construct tridactyl zinc finger proteins that bind to specific sequences was independently developed by the laboratories of Berg (Desjarlais and Berg, 1993), Klug (Choo and Klug, 1994), Pabo (Rebar and Pabo, 1994),and Barbas (Wu et al., 1994). The schemes are very similar; the one depicted here is that of Choo and Klug (1994). The method has been used to obtain proteins that bind to a number of predetermined sequences with high specificity and affinity. The boxed sequence shown in the figure appears 20 base pairs 5′ of the first HpaII (CCGG) site in the LTR sequence of FIG. 2; it is shown for illustrative purposes only (Sequence I.D. No. 2). The actual target sequence will be determined as described in Example 2.

FIG. 10. Organization and transcription of the HBV genome. The viral genome of 3.2 kb is represented by a horizontal line. Positions of methylatable CpG sites are shown by vertical lines. Arrows indicate major viral transcripts. Notice that transcription of each major mRNA initiates in a cluster of CpG sites. CpG methylation has been shown to suppress viral transcription in transgenic animals (Miller and Robinson, 1993; Pourcel et al., 1990).

FIGS. 11A-11B. Targeted methylation of a preselected CpG site.

(A) A diagram of pLS which encodes a lexA-M.SssI fusion protein. A SmaI site is located adjacent ot a lexA binding site in the same plasmid. LexA is predicted to position M.SssI over this site, and preferential methylation of the SmaI site was expected (M.SssI has no intrinsic preference for SmaI sites). The positions of other restriction sites are shown approximately to scale; other features of the plasmid are omitted for the sake of clarity. (B) Data confirms targeted methylation. Odd-numbered lanes contain plasmid that encodes enzymatically active fusion protein. The unmethylated state cuses the DNA to be cleaved to the linear form. Notice that the SmaI site is largely resistant to cleavage in lane 8 (compare to lanes 7 and 10), while the other sites remain sensitive. The methylation-insensitive endonulease NcoI was used as a control. Heavy methylation is observed only at the SmaI site; little or no detectable methylation is seen at SalI and XhoI sites (lanes 4 and 6). Lin: linear unit-length DNA (unmethylated). CCC, covalently closed circular DNA (methylated in lane 88). Lane 11 contains length markers. The diffuse band that migrates above the largest length marker is bacterial chromosomal DNA.

FIG. 12. Construction of a plasmid that performs targeted methylation. pLM9 was constructed by cloning an Lac repressor-M.SssI fusion gene between the Pvull sites of pBluescript. Adjacent to the fusion gene is a synthetic methylation target sequence, LacO-CpGs, which contains the Lac operator (to which Lac repressor binds) and a number of CpG target sites. These are 12 amino acids in the linker between LacI and M.SssI. Expression of the fusion is controlled by the native promoter of IacI gene.

FIG. 13. Expression of LacI-M.SssI fusion in E. coli XL1-Blue [pLM9]. The targeted methyltransferase is marked with an arrow. The antibody used was anti-LacI. An intact fusion protein was produced in good yield. Lane marked pBS contained proteins extracted from cells that contained only the expression vector.

FIG. 14. Targeted methylation demonstrated by bisulfite sequencing analysis. The methylation target shown in FIG. 12 was isolated from cells expressing LacI-M.SssI fusion proteins. The bisulfite sequencing method was used to identify methylated sites. pLM9-1 and -2 are two sister bisulfite cones carrying BS-modified methylation target sequence derived from a pLM9 mutant that has attenuated activity. Methylation is limited to CpG sites in the immediate vicinity of the binding site of the sequence specific DNA binding protein LacI. pBS is the precursor of pLM9 and has no lacI/M.SssI gene; no methylated sites are present. pM is a pBS derivative that encodes fully active (non-targeted) M.SssI gene; all sites are methylated. These data confirm that targeted methylation has been used to direct methylation to CpG sites in the vicinity of the binding site of a sequence-specific DNA binding protein.

FIG. 15. Zinc-finger (Zif) targeted methyltransferases. The above Zif fusion constructs have been made and expression of the appropriate fusion proteins has been confirmed by immunoblot and methylation analysis. The coding regions have also been transferred into the mammalian expression vector pcDNA3.1/His/A.

FIG. 16. HIV-1 LTR constructs used to analyze targeted methylation. Plasmid PLTR-CAT (top) contains the U3 region and 80% of the R region of the HIV-1 proviral long terminal repeat (LTR) driving expression of the chloramphenicol acetyltransferase (CAT) reporter gene. These elements contain the HIV-1 core promoter, enhancer, negative regulatory element, and trans-activated region (TAR). These elements also contains 10 CpG dinucleotides (shown as circles above the LTR), which are targets for methylation. Plasmid pLG-luc (bottom) contains the entire proviral LTR and gag leader sequence (GLS) driving expression of the firefly luciferase reporter gene. The 150 bp GLS, which lies between the LTR and the first viral gene, contains an additional 14 CpG dinucleotides and has been shown to be important for viral expression. The circles are shaded (see box) to indicate the conservation of the individual CpG sites in different HIV-1 sequences of class B, the class affecting the vast majority of infected individuals in the United States and Western Europe. Transcription factor binding sites are labeled below the LTR and hatched when they contain a CpG site. The LTR is flanked by a short region of human genomic DNA present in the original HIV clone.

FIG. 17. Complete suppression of HIV-1 transcription by LTR methylation. Methods: pLTR-CAT was treated with M.SssI to methylate all CpG sites. Methylated or unmethylated pLTR-CAT was cotransfected with unmethylated pLG-luc into HLtat cells and lysates were prepared at the times indicated. To control for the efficiency of transfection and recovery, volumes of lysate containing equivalent luciferase activity were assayed for CAT activity using the FASTCAT assay kit. Reaction products were quantitated using NIH Image. “CAT Activity” indicates conversion of 1-deoxychloramphenicol to 3-acetyl-1-deoxychloramphenicol, normalized to arbitrary units. Control cells were transfected with pLG-luc only and harvested 48 hours post-transfection.

### DETAILED DESCRIPTION OF THE INVENTION

The present invention provides for a chimeric protein which comprises a mutated DNA methyltransferase portion and a DNA binding protein portion that binds sufficiently close to a promoter sequence of a target gene, which promoter sequence contains a methylation site, to specifically methylate the site and inhibit activity of the promoter and thus inhibit expression of the target gene.

As used herein, “chimeric protein” is a protein including at least two portions of two proteins positioned adjacent to each other so that the coding sequences of both portions are in frame and can be translated into one polypeptide. The chimeric protein may include a portion of a DNA binding protein and a portion of a mutated or wild type DNA methyltransferase protein. The two portions of the chimeric protein may be linked with intermediate sequences of 8,12,16 or 20 codons expressed as part of the chimeric protein.

As used herein, “adjacent” includes two sequences that are positioned directly next to one another so that the codons of each sequence are in frame and contiguous. “Adjacent” also includes two sequences that are nearby one another linked to intermediate sequences and are in frame and can be translated into one polypeptide.

As used herein “linked to” encompasses a means of joining two separate portions of a protein, each of which may have a separate function. The means of joining the functional portions of the protein may include a covalent or noncovalent association between functional polypeptides via an organic substance, an inorganic substance, a nucleotide, a polynucleotide, a peptide nucleic acid, a peptide, a triplex peptide, an electrostatic means, a triplex nucleic acid, or a self-assembling biomaterial.

As used herein, “DNA methyltransferase” is a protein which is capable of methylating a particular DNA sequence, which particular DNA sequence may be -CpG-. This protein may be a mutated DNA methyltransferase, a wild type DNA methyltransferase, a naturally occurring DNA methyltransferase, a variant of a naturally occurring DNA methyltransferase, a truncated DNA methyltransferase, or a segment of a DNA methyltransferase which is capable of methylating DNA. The DNA methyltransferase may include mammalian DNA methyltransferase, bacterial DNA methyltransferase, M.SssI DNA methyltransferase and other proteins or polypeptides that have the capability of methylating DNA.

As used herein, “mutated DNA methyltransferase protein portion” is a segment of a DNA methyltransferase protein or polypeptide containing an amino acid sequence which is different from the wild type DNA methyltransferase amino acid sequence. A mutated DNA methyltransferase protein portion may include a segment of a naturally occurring variant of the wild type DNA methyltransferase protein, a segment of a DNA methyltransferase with a sequence altered from the wild type sequence, a truncated protein or portion of protein or polypeptide that is capable of methylating DNA. This mutated DNA methyltransferase may be altered at one or more of its amino acids and retains part of the wild type methyltransferase activity, although the activity of the mutated DNA methyltransferase protein portion may be attenuated activity.

As used herein, “DNA binding protein portion” is a segment of a DNA binding protein or polypeptide capable of specifically binding to a particular DNA sequence. The binding is specific to a particular DNA sequence site. The DNA binding protein portion may include a truncated segment of a DNA binding protein or a fragment of a DNA binding protein.

As used herein, “binds sufficiently close” means the contacting of a DNA molecule by a protein at a position on the DNA molecule near enough to a predetermined methylation site on the DNA molecule to allow proper functioning of the protein and allow specific methylation of the predetermined methylation site.

As used herein, “a promoter sequence of a target gene” is at least a portion of a non-coding DNA sequence which directs the expression of the target gene. The portion of the non-coding DNA sequence may be in the 5′-prime direction or in the 3′-prime direction from the coding region of the target gene. The portion of the non-coding DNA sequence may be located in an intron of the target gene.

The promoter sequence of the target gene may be a 5′ long terminal repeat sequence of a human immunodeficiency virus-1 proviral DNA. The target gene may be a retroviral gene, an adenoviral gene, a foamy viral gene, a parvo viral gene, a foreign gene expressed in a cell, an overexpressed gene, or a misexpressed gene.

As used herein “specifically methylate” means to bond a methyl group to a methylation site in a DNA sequence, which methylation site may be -CpG-, wherein the methylation is restricted to particular methylation site(s) and the methylation is not random.

In one embodiment of this invention, the chimeric protein may be a zinc three-finger DNA binding polypeptide linked to a CpG-specific DNA methyltransferase protein portion. The chimeric protein may comprise a mutated Lex A DNA binding protein or polypeptide linked to a cytosine methyltransferase protein portion or polypeptide.

The chimeric protein may include at least a portion of a mutated M.SssI DNA methyltransferase protein or at least a portion of a mutated mammalian DNA methyltransferase.

In one embodiment of this invention, an expression vector may code for the chimeric protein described hereinabove. The expression vector may be a replicable vector and may include a pLS vector, a prokaryotic expression vector, a yeast expression vector, a baculovirus expression vector, a mammalian expression vector, or an episomal mammalian expression vector.

As used herein, “expression vector” is a nucleic acid molecule which is capable of having a coding sequence of DNA or RNA ligated into it and then expressing that sequence when introduced into an appropriate host cell. An expression vector may be a mammalian expression vector or a prokaryotic expression vector.

Another embodiment of this invention is a method for inhibiting the expression of a target gene which includes contacting a promoter sequence of the target gene with the chimeric protein described hereinabove, so as to specifically methylate the promoter sequence of the target gene thus inhibiting expression of the target gene. In this embodiment, the target gene may be an endogenous target gene which is native to a cell or a foreign target gene. The foreign gene may be a retroviral target gene or a viral target gene.

The target gene in this embodiment may be associated with a cancer, a central nervous system disorder, a blood disorder, a metabolic disorder, a cardiovascular disorder, an autoimmune disorder, or an inflammatory disorder. The cancer may be acute lymphocytic leukemia, acute myelogenous leukemia, B-cell lymphoma, lung cancer, breast cancer, ovarian cancer, prostate cancer, lymphoma, Hodgkin's disease, malignant melanoma, neuroblastoma, renal cell carcinoma or squamous cell carcinoma. The central nervous system disorder may be Alzheimer's disease, Down's syndrome, Parkinson's disease, Huntington's disease, schizophrenia, or multiple sclerosis. The infectious disease may be cytomegalovirus, herpes simplex virus, human immunodeficiency virus, AIDS, papillomavirus, influenza, candida albicans, mycobacteria, septic shock, or associated with a gram negative bacteria. The blood disorder may be anemia, hemoglobinopathies, sickle cell anemia, or hemophilia. The cardiovascular disorder may be familial hypercholesterolemia, atherosclerosis, or renin/angiotensin control disorder.

The metabolic disorder may be ADA, deficient SCID, diabetes, cystic fibrosis, Gaucher's disease, galactosemia, growth hormone deficiency, inherited emphysema, Lesch-Nyhan disease, liver failure, muscular dystrophy, phenylketonuria, or Tay-Sachs disease. The autoimmune disorder may be arthritis, psoriasis, HIV, or atopic dermatitis. The inflammatory disorder may be acute pancreatitis, irritable bowel syndrome, Chrone's disease or an allergic disorder.

In one embodiment of the subject invention promoter sequences of the target genes listed below may be therapeutic target genes of the subject invention: dominant oncogenes, c-MYB, c-MYC, c-SIS, ERB A, ERB B, ERB B-1, ERB B-2, HER-2/NEU, c-SRC, c-YES, c-FPS, c-FES, c-FOS, c-JUN, c-ROS, c-ABL, c-FGR, c-MOS, C-RAS, C-RAF, C-MET, C-ETS, BCL-1, BCL-2, ETS, c-FMS, c-FES, c-BLK, TCL-1, TCL-2, TCL-3, TCL-5, ALL-1/HRX/MLL, PML promoter in PML/RAR-α fusion, RAR-A promoter in RAR-a/PML fusion, and NF-1.

Genes that are overexpressed in cancer cells are also target genes of the subject invention. Inhibiting the expression of these target genes may reduce tumorigenesis and/or metastasis and invasion. Cancer related genes include: collagenase 92 Kd Type 4, collagenase 72 Kd Type 4, osteopontin, calcyclin, fibroblast growth factor, epidermal growth factor, matrilysin and stromolysin.

Viruses that establish chronic infections and which are involved in cancer or chronic diseases are also target genes of the subject invention. Virus that have possible target genes include hepatitis C, hepatitis B, varicella, herpes simplex types I and II, Epstein-Barr virus, cytomegalovirus, JC virus and BK virus.

In a preferred embodiment of the subject invention, the promoter sequence of viral protein X and the promoter sequence of pre-S2/5 of the hepatitis B virus may be target genes.

In another embodiment of the subject invention, genes whose expression is involved in neurodegenerative disease may be target genes of the subject invention. These target genes may include beta amyloid precursor protein and the prion protein both of which are shown to be involved in Alzheimer's disease. In another embodiment of the subject invention, genes which are involved in the etiology of acromegaly may be target genes of the subject invention. These target genes may include somatostatin, growth-hormone releasing hormone and constitutive G-stimulatory protein mutants.

The target gene may be in a cell. The cell may be a bacterial cell, an animal cell or a plant cell. The cell may be a eukaryotic cell, a prokaryotic cell, a virus packaging cell, a somatic cell, a germ cell, a neuronal cell, a myocyte, a T lymphocyte, a CD4+ cell, a tumor cell, a CD4+ cell, or a stem cell.

The chimeric protein may be a mutated Lex A DNA binding protein portion or polypeptide linked to a cytosine DNA methyltransferase protein portion or polypeptide.

The DNA methyltransferase protein portion may be M.SssI DNA methyltransferase protein or the mammalian DNA methyltransferase protein. The chimeric protein may include a tridactyl zinc finger protein or polypeptide capable of specifically binding the HIV-1 5′ LTR nucleic acid sequence linked to a cytosine methyltransferase polypeptide. The cytosine DNA methyltransferase polypeptide may be a M. SssI DNA methyltransferase protein or polypeptide or at least a portion of the mammalian DNA methyltransferase protein.

The contacting may be by means of liposome mediated delivery, retroviral delivery, gene bombardment, electroporation, electronic pulse delivery, air-gun injection of a nucleic acid-coated pellet, a self-assembling nanocrystalline composition, a cytofectin analog, condensation of the nucleic acid, receptor mediated gene transfer, glycosylated macromolecular carriers, polar (glyco) lipids, (glyco)peptides, synthetic polymers, a triplex nucleic acid, naked nucleic acid transfer, particle-mediated nucleic acid transfer or cationic precipitation.

A further embodiment of this invention is a method for inhibiting expression of a target gene in a multicellular organism which includes contacting a promoter sequence of the target gene with the chimeric protein described hereinabove or a nucleic acid molecule encoding the chimeric protein, so as to specifically methylate the promoter sequence of the target gene and thus inhibit expression of the target gene in the multicellular organism.

The multicellular organism may be a plant, an animal or a human. The plant may be an alfalfa plant, a broccoli plant, a rapeseed plant, a carrot plant, a chicory plant, a coffee plant, a cucurbita plant, a euromelon plant, a potato plant, a raspberry plant, a sunflower plant, a tomato plant, or a wheat plant.

The administration of the chimeric protein to the multicellular organism may comprise intralesional, intraperitoneal, intramuscular or intravenous injection; liposome-mediated delivery; viral infection; gene bombardment; topical, nasal, oral, anal, ocular or otic delivery. The viral infection may be via a non-integrating, replication-defective virus. The virus may comprise a replication-defective HIV-1 provirus, a retroviral vector, an adeno-associated virus, a N2 retroviral vector, a SIM retroviral vector, a LNL6 vector, a LXSN vector or a MMuLV retroviral vector.

The process by which a plant or animal is rendered resistant to viral infection comprises introducing into the plant or animal a construct which on translation gives rise to the above-mentioned chimeric protein. The introduction of the nucleic acid molecule is accomplished by genetic transformation of a part of the plant by a DNA sequence coding for the nucleic acid molecule, followed by the regeneration of a transgenic plant. The transformation is carried out by the intermediary of Agrobacterium tumefaciens or Agrobacterium rhizogenes. (U.S. Pat. Nos. 5,107,065 and 5,188,958).

The present invention is further directed to a DNA construct for a plant, the construct comprising a genetic sequence and a promoter capable of directing expression of the genetic sequences wherein the genetic sequence on expression provides at least a portion of a sequence specific DNA binding protein linked to at least a portion of a mutated DNA methyltransferase protein. The DNA construct may further be part of a DNA transfer vector suitable for transferring the DNA construct into a plant cell and insertion into a plant genome. In an embodiment of the present invention, the DNA construct is carried by broad host range plasmid pGA470 which is capable of transformation into plant cells using Agrobacterium. The present invention, however, extends to other means of transfer such as genetic bullets (e.g. DNA-coated tungsten particles, high-velocity micro projectile bombardment) and electroporation amongst others (Maliga, 1993; Bryant, 1992; or Shimamoto, 1989).

The gene encoding the chimeric protein, having been introduced into the nonhuman mammal, or an ancestor of the nonhuman mammal at the single cell stage or an embryonic stage, is linked to a promoter and integrated into the genome of the nonhuman mammal. One skilled in the art would be familiar with the experimental methods necessary to produce a transgenic mammal, as described in Leder et al., U.S. Pat. No. 4,736,866 and Krimpenfort and Berns, U.S. Pat. No. 5,175,384 and Wagner and Chen, U.S. Pat. No. 5,175,385.

A further embodiment of the subject invention is a method of treating a subject infected with a virus which comprises administering to the subject a therapeutic composition comprising a chimeric protein, or a nucleic acid molecule encoding the chimeric protein, capable of sequence specific methylation of a site(s) in a promoter sequence of a target viral gene under suitable conditions so as to specifically methylate the promoter sequence of the target gene thereby inhibiting expression of the target gene and thus treating the subject infected with the virus. Examples of viruses are listed herein.

The therapeutic composition may comprise a nucleic acid encoding at least a portion of a mutated Lex A DNA binding protein or polypeptide linked to at least a portion of a mutated DNA methyltransferase protein or polypeptide. The therapeutic composition may be a nucleic acid encoding a tridactyl zinc finger DNA binding protein or polypeptide capable of specifically binding the HIV-1 5′ LTR nucleic acid sequence linked to at least a portion of a mutated DNA methyltransferase protein or polypeptide. The therapeutic composition may comprise a nucleic acid encoding at least a portion of a sequence specific DNA binding protein or polypeptide linked to a mutated DNA methyltransferase protein or polypeptide.

The subject receiving the therapeutic composition of this invention may be a human or a non-human mammal.

The therapeutic composition may comprise a replicable expression vector chosen from the group consisting of a pLS vector, a prokaryotic expression vector, a yeast expression vector, a baculovirus expression vector, a mammalian expression vector, and an episomal mammalian expression vector. The administration may comprise intralesional, intraperitoneal, intramuscular or intravenous injection; liposome-mediated delivery; viral infection; gene bombardment; topical, nasal, oral, anal, ocular or otic delivery. The viral infection may be via a non-integrating, replication-defective virus.

In one preferred embodiment of the method above the nucleic acid molecule encoding a chimeric protein is incorporated into a liposome to allow for administration to the subject. Methods of incorporation of nucleic acid molecules into liposomes are well known to those of ordinary skill in the art. In another embodiment of this method, the nucleic acid encoding the chimeric protein may be delivered via transfection, injection, or viral infection. There are several protocols for human gene therapy which have been approved for use by the Recombinant DNA Advisory Committee (RAC) which conform to a general protocol of target cell infection and administration of transfected cells (see for example, Blaese, R. M., et al., 1990; Anderson, W. F., 1992; Culver, K. W. et al., 1991). In addition, U.S. Pat. No. 5,399,346 (Anderson, W. F. et al., issued Mar. 21, 1995) describes procedures for retroviral gene transfer. The contents of these support references -are incorporated in their entirety into the subject application. Retroviral-mediated gene transfer requires target cells which are undergoing cell division in order to achieve stable integration hence, cells are collected from a subject often by removing blood or bone marrow.

Several methods have been developed over the last decade for the transduction of genes into mammalian cells for potential use in gene therapy. In addition to direct use of plasmid DNA to transfer genes, episomal vectors, retroviruses, adenoviruses, parvoviruses, and herpesviruses have been used (Anderson et al., 1995; Mulligan, 1993; The contents of which are incorporated in their entirety into the subject application). For transfer of genes into cells ex vivo and subsequent reintroduction into a host, as would be most feasible in immunodeficiency patients, retroviruses have been the vectors of choice.

A separately preferred embodiment of the invention is a replicable expression vector encoding a chimeric protein capable of sequence specific DNA methylation.

The replicable expression vector may be chosen from the group consisting of a pLS vector, a prokaryotic expression vector, a yeast expression vector, a baculovirus expression vector, a mammalian expression vector, and an episomal mammalian expression vector. The agent may comprise a mutated Lex A binding polypeptide linked to a methyltransferase polypeptide. The agent may comprise a tridactyl zinc finger polypeptide that specifically binds to the HIV-1 5′LTR nucleic acid sequence linked to a methyltransferase polypeptide.

Another embodiment of the invention is a pharmaceutical composition comprising a therapeutically effective amount of the replicable vector described hereinabove and a pharmaceutically acceptable carrier. The carrier may comprise a diluent. The pharmaceutically acceptable carrier may be an aerosol, intravenous, oral or topical carrier and is further described herein.

Another embodiment of the subject invention is a method of obtaining an expression vector including, DNA encoding a desired chimeric protein that includes at least a portion of a mutated DNA methyltransferase protein and at least a portion of a DNA binding protein such chimeric protein being capable of inhibiting the expression of a target gene which comprises: (a) obtaining a population of expression vectors each of which expresses a chimeric protein including (1) a DNA corresponding to an endogenous promoter sequence of the target gene, which promoter contains at least one sequence specific methylation site; (2) a DNA encoding a mutated DNA methyltransferase; and (3) a DNA encoding a DNA binding protein, position adjacent the DNA of (2), DNA (2) and (1) being so positioned in the expression vectors as to permit expression of the chimeric proteins; (b) introducing the population of vectors from step (a) into an appropriate host under conditions such that the chimeric proteins are expressed and methylate DNA; ( c) isolating the population of vectors from step (b) from the host; (d) treating the population of vectors from step ( c) with a suitable restriction endonuclease so as to digest the DNA at a specific site in the promoter if the site is not methylated; (e) introducing the population from step (d) into an appropriate host, which hosts have the property that they degrade DNA which has been non-specifically methylated at sites other than the sequence specific methylation site contained in the promoter; and (f) culturing the hosts from step (e) under conditions such that vectors which have not been degraded express either a mutated or a non-mutated form of the chimeric protein, thus obtaining at least one nucleic acid encoding a mutated form of a chimeric DNA methyltransferase/DNA binding protein capable of inhibiting the expression of the target gene.

As used herein, “desired” includes optimal properties of a chimeric protein as described hereinabove. The properties include specifically methylating a DNA sequence in a promoter sequence of a target gene and inhibiting the activity of the promoter and thus inhibiting expression of the target gene.

In the practice of any of the methods of the invention or preparation of any of the pharmaceutical compositions an “effective amount” is an amount which is effective to produce the sequence specific methyltransferase and inactivation of the target gene. Accordingly, the effective amount will vary with the subject being treated, as well as the condition to be treated. For the purposes of this invention, the methods of administration are to include, but are not limited to, administration cutaneously, subcutaneously, intravenously, parenterally, orally, topically, or by aerosol.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

Also comprehended by the invention are pharmaceutical compositions comprising therapeutically effective amounts of polypeptide products of the invention together with suitable diluents, preservatives, solubilizers, emulsifiers, adjuvants and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. The choice of compositions will depend on the physical and chemical properties of the protein. For example, a product derived from a membrane-bound form of a protein may require a formulation containing detergent. Controlled or sustained release compositions include formulation in lipophilic depots (e.g., fatty acids, waxes, oils). Also comprehended by the invention are particulate compositions coated with polymers (e.g., poloxamers or poloxamines) and coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

Another embodiment of the subject invention is a transgenic non-human mammal whose somatic and germ cells contain and express a gene coding for a desired chimeric protein which is capable of inhibiting the expression of a target gene, the gene having been stably introduced into the non-human mammal at the single cell stage or an embryonic stage, and wherein the gene is linked to a promoter and integrated into the genome of the non-human mammal. The desired chimeric protein may be the desired chimeric protein described hereinabove. One skilled in the art would be familiar with the experimental methods necessary to produce a transgenic mammal, as described in Leder et al., U.S. Pat. No. 4,736,866 and Krimpenfort and Berns, U.S. Pat. No. 5,175,384 and Wagner and Chen, U.S. Pat. No. 5,175,385.

If it were possible to stimulate de novo methylation of HIV proviral DNA, then clonal inheritance of methylation patterns would render the inactivation essentially irreversible. As described herein, this irreversible inactivation is the basis of a therapeutic approach that has practical advantages over alternative molecular biological approaches (including antisense RNA, ribozymes, targeted nucleases, and intracellular anti-HIV antibodies; reviewed by Volberding, 1995). Provirus silencing by targeted cytosine methylation stimulates an existing host defense system and as an analogy, has the virtues of a vaccine: the clonal inheritance of methylation patterns is like the immunological memory, and the strong suppression of transcription is like the immune defense system in that both perturb the function of particular gene products.

It is important to point out that the therapeutic agent that methylates the target sequence need be present only transiently; the new methylation pattern would be propagated indefinitely by the cellular DNA methylating system (Bestor, 1990; Bestor, 1992; Bestor and Coxon, 1993; Bestor and Verdine, 1994). The alternate molecular biological approaches to medical therapies mentioned above, require indefinite expression of the agent. This has proven to be difficult in practice (Challita and Kohn, 1994) and may be toxic or may provoke a harmful immune response. An additional complication of long-term expression is insertional mutagenesis, while inactivation by targeted methylation as described herein may involve delivery of the agent by a nonintegrating, replication-defective virus that establishes an abortive infection. None of the alternative molecular biological therapies discussed above has these advantages.

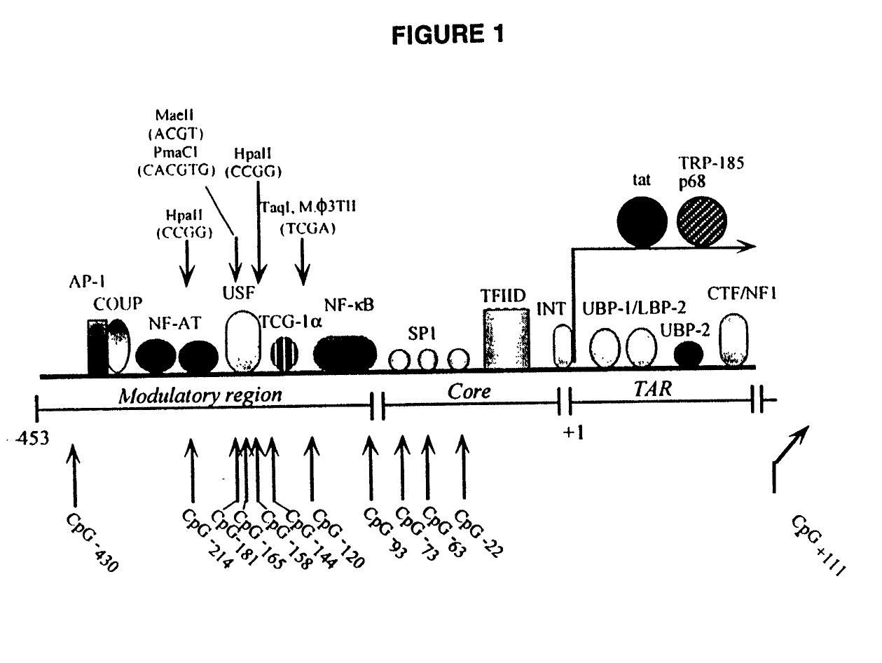

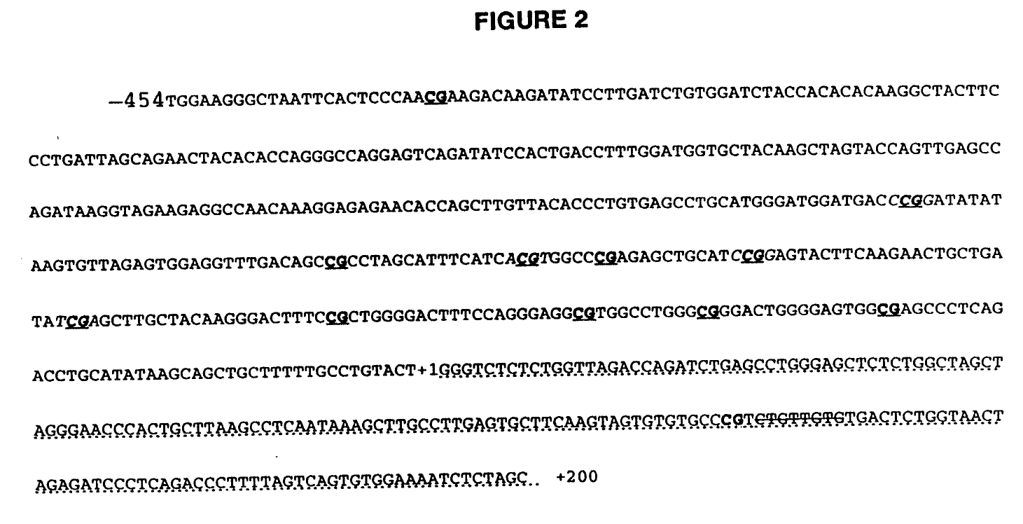

The 5′ LTR of HIV-1 may be inactivated by targeted cytosine methylation. As shown in FIG. 1 below, the LTR that directs transcription of the provirus is rich in binding sites for transcription stimulatory proteins (Nabel, 1993; Garcia and Gaynor, 1994) and rich in CpG sites, the predominant site of methylation in vertebrates. It is also well-established that methylation of only a few of the CpG sites in the 5′ LTR represses transcription to nearly undetectable levels (Bednarik et al., 1990; Joel et al., 1993, reviewed by Bednarik, 1992), but the specific, critical sites that mediate this effect have not been identified.

Mammalian promoters are silenced by cytosine-5 methylation at 5′-CpG-3′ dinucleotides. The promoter within the 5′ LTR of HIV-1 may be silenced by directing enzymatic methylation to critical CpG sites. Because methylation patterns are subject to clonal inheritance, the silencing is essentially irreversible.

The subject invention encompasses but is not limited to the following additional embodiments.

1. Methylation of a predetermined CpG site. In one embodiment of the subject invention a sequence-specific DNA binding protein may be fused to a DNA cytosine methyltransferase, and mutant fusion proteins whose methyltransferase activity is dependent on the DNA binding protein may be selected from combinatorial libraries. DNA binding protein/DNA methyltransferase chimeras that methylate only those CpG sites immediately adjacent to the binding site may be obtained by means of a novel cyclic in vivo/in vitro selection protocol. An attenuated version of the CpG-specific DNA methyltransferase M.SssI may be constructed in this way. In vitro selection requires resistance to methylation-sensitive restriction endonuclease cleavage at the target site, and in vivo selection removes plasmid molecules methylated at non-specific sites; this second step involves growth in McrBC_{═}⁺ strains of E. coli, which degrade heavily methylated DNA.

2. Identification of those CpG sites in the HIV-1 5′ LTR whose methylation produces maximal repression of transcription. The documented inhibitory effects of methylation on HIV-1 transcription are likely to be exerted through a subset of the 11 CpG sites in the 5′ LTR of HIV-1. In a further embodiment of the subject invention, these critical CpG sites may be identified by transcription assays with test constructs that bear singly methylated CpG sites; each of the 11 CpG sites can be tested individually. The information obtained may be important for the selection of target sites in Examples 3-5.

3. Design, selection, and affinity maturation of zinc finger-DNA methyltransferase chimeras that methylate critical CpG sites in the HIV 5′ LTR. In another embodiment of the subject invention, zinc finger modules may be catenated in novel combinations to yield proteins that bind with high affinity and specificity to predetermined sequences. Zinc finger proteins that bind to sites adjacent to critical CpG sites (identified in Example 2) may be selected from combinatorial expression libraries and fused to an attenuated CpG-specific DNA methyltransferase (developed in Example 1). Further rounds of cyclic in vivo/in vitro selection (as developed in Example 1) may be used to obtain variants that methylate only CpG sites adjacent to the binding site of the zinc finger moiety.

4. Inhibition of HIV-1 5′ LTR-dependent transcription in cultured human cells that express zinc finger-DNA methyl-transferase fusion proteins of novel and predetermined specificity. Example 3 yields constructs that methylate critical sites in the HIV-1 5′ LTR. In a further embodiment of the subject invention, these constructs may be introduced into cultured human cells that express a reporter gene driven by the HIV-1 5′ LTR as a means of testing the inhibitory effects of targeted de novo methylation.

5. Inhibition of HIV-1 replication in human T lymphocytes productively infected with HIV-1. In another embodiment of the subject invention, constructs that prove effective in the studies of Example 4 may be introduced into HIV-1 producing human T lymphocytes, and methylation of the target sequence and its effect on virus production can be measured directly. The methylating construct may be delivered on a plasmid vector or as part of a recombinant adeno-associated virus (AAV). These embodiments suggest a high level of usefulness of targeted methylation in the control of HIV-1 proliferation, and the development of the methylating construct into a therapeutic agent.

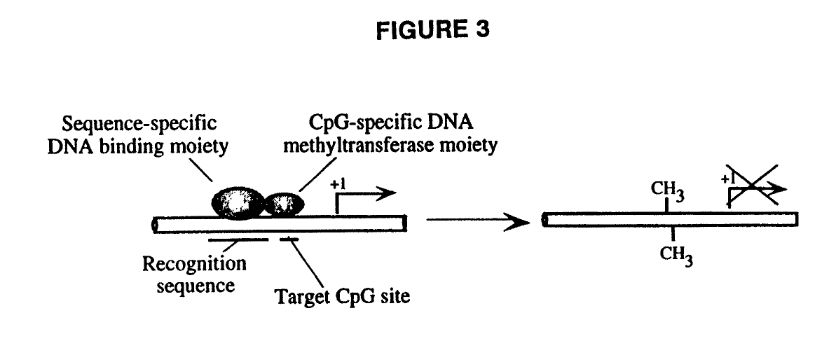

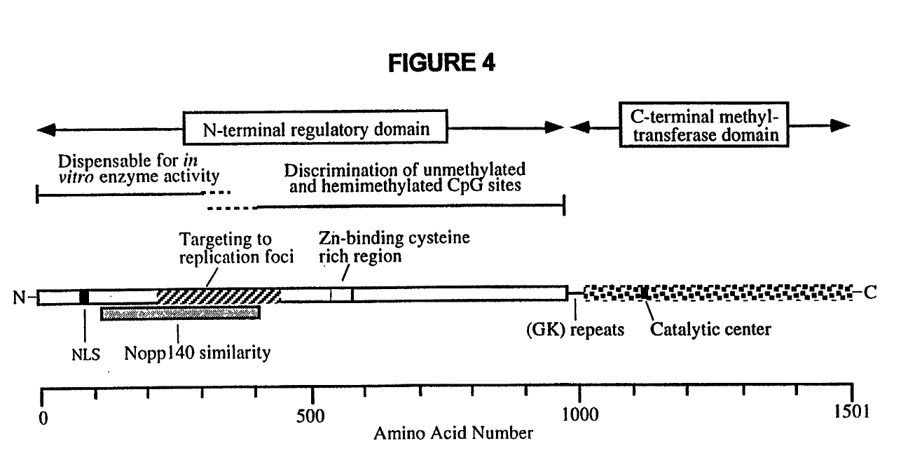

Direction of a DNA methyltransferase to any CpG site within HIV-1 regulatory sequences, may result in the silencing of the provirus dependent upon the specific zinc finger protein/DNA methyltransferase chimera. Zinc finger motifs have been shown to be remarkably versatile DNA binding structures (Choo and Klug, 1994); they are independently-folded protein modules that recognize the edges of 3 base pairs in the major groove. Over 1,300 zinc fingers have been identified, and the binding specificity of a large number has been determined (Choo et al., 1995). Several laboratories have succeeded in assembling zinc finger modules in novel combinations; these designed proteins have been subjected to refinement by phage-display selection, and variants that bind to novel predetermined sequences with high specificity and nanomolar affinity have been obtained by several laboratories (Choo et al., 1994; Wu et al, 1995; Desjarlais and Berg, 1994; Rebar and Pabo, 1994). These small proteins contain tandem arrays of three zinc finger modules and bind to 9 bp recognition sequences. Klug and colleagues were able to show that a zinc finger that was targeted to the BCR-ABL breakpoint sequence could bind to and suppress transcription of the BCR-ABL gene in cultured human leukemic cells (Choo et al., 1994). A zinc finger protein designed to bind to a sequence adjacent to a CpG site in the HIV-1 5′ LTR can be fused to an attenuated CpG-specific DNA methyltransferase, thereby achieving efficient, selective methylation of that CpG site. A DNA methyltransferase with the desired binding properties has been cloned from a Spiroplasma species; this enzyme, known as M.SssI, has the same sequence specificity as the mammalian DNA methyltransferase (CpG) (as cloned by Bestor; Genbank Accession No. X14805), but a higher turnover number and no requirement for hemimethylated substrates (Renbaum and Razin, 1992). The strategy is shown in FIG. 3.

One embodiment of this invention is the development and therapeutic application of zinc finger protein/DNA methyltransferase chimeras or agents whose general organization is similar to that of mammalian DNA methyltransferase. These agents selectively methylate and inactivate the 5′ LTR of HIV-1. Such agents have considerable promise as therapeutic agents for retroviral diseases, and for other applications where selective inactivation of a promoter sequence is desired.

The zinc finger protein/DNA methyltransferase chimera of this invention is similar in design to mammalian DNA methyltransferase, which has been purified (Bestor and Ingram, 1983; Bestor and Ingram, 1985a), characterized in terms of biochemical activities (Bestor and Ingram, 1985; Bestor, 1987), the cDNA that encodes the enzyme cloned (Bestor, 1988; Genbank Accession No. X14805; Bestor et al., 1988), and the protein demonstrated to be essential for mouse development in a gene disruption study (Li et al., 1992; Li et al., 1993). As shown below, mammalian DNA methyltransferase has an N-terminal regulatory domain and a C-terminal catalytic domain that closely resembles bacterial restriction methyltransferases. For-a recent review of the structure and function of mammalian DNA methyltransferase, see Bestor and Verdine (1994). The agents which are embodiments of this invention differ from mammalian DNA methyltransferase in that the regulatory domain targets the catalytic domain to predetermined sequences. The regulatory region of mammalian DNA methyltransferase suppresses the methylation of previously unmethylated sites (Bestor, 1992), and directs the protein to sites of new DNA synthesis in S (synthesis) phase nuclei (Leonhardt et al., 1992). The targeted methylation which is an embodiment of this invention is grounded in the knowledge of the basic biology of the system that establishes and maintains methylation patterns in the mammalian genome.

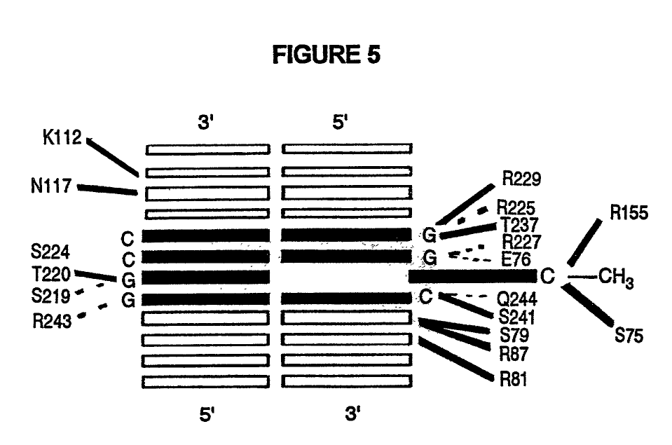

The expression construct pCAL7 directs the expression of the DNA methyltransferase M.SssI (Renbaum and Razin, 1992). This enzyme in present in Spiroplasma species and has the same sequence specificity as mammalian DNA methyltransferase (CpG), but a higher turnover number and no requirement for hemimethylated substrates (Renbaum and Razin, 1992). The C-terminal domain of mammalian DNA methyltransferase also has CpG-specific DNA methyltransferase activity and may also be used in practicing this invention, although k_{cat} is much lower than that of M.SssI (Bestor, 1992; Renbaum and Razin, 1992). The plasmid construct described in Example 1 may be confirmed by restriction analysis and is described in full in the following section and depicted in FIG. 5. This construct embodies (in a simple form) most of the principles of the targeted methylation approach described herein.

Table 1 shown below, offers data which validates the selection against indiscriminate methylation via McrBC restriction of methylated DNA (Table 1).

TABLE 1
Selection against nonspecific methylation in
McrBC+ E. coli. pSE4 is an unmethylated control plasmid;
pCAL7 directs synthesis of M.SssI (Renbaum et al., 1992)
under the control of the ₚtac promoter. Plasmids were
introduced into the indicated bacterial strains (Raleigh,
1992; Sutherland et al., 1992) by electroporation, and
transformation efficiencies determined by colony counts on
selective media. The values shown are conservative, as IPTG
induction was not used in this case, and the plasmid was
only partially methylated as a result of basal expression
from the ₚtac promoter; measurements of HpaII resistance
indicated <50% methylation. IPTG treatment increased the
methylation level and increased restriction from a factor of
˜10³ to 10⁴.

| E. coil strain | Input plasmid | |
|---|---|---|
| (Mcr genotype) | (methylation status) | CFU μg DNA⁻¹ |
| ER1381 (McrBC⁺) | pSE4 (−) | 1.1 × 10⁹ |
| ER1793 (McrBC⁻) | pSE4 (−) | 1.7 × 10⁹ |
| ER1381 (McrBC⁺) | pCAL7 (+) | 6 × 10⁵ |
| ER1793 (McrBC⁻) | pCAL7 (+) | 8 × 10⁸ |

Promoter Inactivation Via Targeted Cytosine Methylation.

A lambda repressor-integrase fusion protein has been shown to direct HIV-1 integration to sites in the vicinity of lambda operators (Bushman, 1994). Through an analogous mechanism, a chimeric DNA binding protein/DNA methyltransferase will cause preferential methylation of sites in the vicinity of the recognition sequence of the DNA binding protein through a very large increase in the local concentration of DNA methyltransferase. As shown in FIG. 3, this may be used to selectively methylate and silence predetermined promoter sequences.

Crystallography data have shown that DNA cytosine methyltransferases make base-specific contacts in the major groove, and numerous sequence-independent contacts with the DNA backbone (Bestor and Verdine, 1994). A diagram of these contacts is shown in FIG. 5.

Mutations of residues involved in the backbone contacts lowers the affinity of the enzyme for DNA and reduces the rate of methyl transfer. However, tethering the weakened DNA methyltransferase to a sequence-specific DNA binding protein greatly increases the concentration of the DNA methyltransferase in the vicinity of the binding site, thus offsetting the effects of reduced affinity. This results in a chimeric protein that methylates sites only in the immediate vicinity of the recognition sequence of the DNA binding protein. Toxicity due to methylation of collateral CpG sites is obviated. Rather than estimating the nature of mutations that would yield the desired result, proteins with the desired properties may be selected from large combinatorial libraries. The design and selection of such chimeric proteins, and their development into potential therapeutic agents, is an embodiment of the subject invention. The 5′ LTR of HIV-1 may be one target of inactivation, and a rational, step-wise approach to the this is described below in the Experimental Details.

Gene Inactivation by Targeted Methylation

1. Additional Embodiments of Methylating Agents.

As mentioned above, the zinc finger/DNA methyltransferase chimeras discussed and described herein are analogous in structure to mammalian DNA methyltransferase, the enzyme that normally establishes and maintains methylation patterns in the genome. This enzyme has a zinc binding site that has been shown to participate in the discrimination of unmethylated and hemimethylated CpG sites (Bestor, 1992). Replacement of this zinc-binding region with a tridactyl finger region of known binding specificity confers a new de novo specificity on the enzyme. Such an enzyme may be less immunogenic, and the efficiency of methylation may be increased because the coupling of DNA replication and methylation would be restored (Leonhardt et al., 1992). New generations of increasingly effective methylating agents may be developed using this approach.

2. Delivery of the Methylating Agent

Described herein is a new therapeutic agent. Many delivery agents are described and encompass embodiments of this invention which are technically sufficient to allow delivery to the target cell population. The AAV transducing vector used in the experiments of Example 5 has many advantages, although other transducing vectors with superior properties are also encompassed in this invention. Delivery of the methylating agent to a target cell population has no additional difficulties over those associated with other molecular biological therapeutics.

As pointed out earlier, in favorable cases the methylating agent need be present only transiently,, since the new methylation pattern is subject to clonal inheritance, and gene silencing mediated by this agent is essentially irreversible. This advantage is not enjoyed by alternative molecular biological therapeutics. In cases of recurrent infection or relapse due to silencing of an inadequate fraction of the proviral load, further treatments with the original agent or an agent that recognizes a different critical CpG site may be undertaken. The highly versatile nature of the methylating agents makes such an approach more favorable than the extant molecular biological therapeutic agents.

3. Prophylaxis in Humans and Animals

Targeted methylation may also be applied to prophylaxis and the prevention of recurrent infections. One promising route involves a zinc finger/DNA methyltransferase chimera that resides in the cytoplasm by virtue of the lack of nuclear localization signals, and which methylates incoming viral DNA during its transit through the cytoplasm. Previous work has demonstrated that retroviral DNA is accessible to nucleases (and therefore to methyltransferases of similar size) while in the cytoplasm (Bowerman et al., 1989), and methylation prior to integration would yield a provirus that is transcriptionally silenced from the time of integration. Long-term, stable expression of such a construct could protect recurring infections with HIV-1 or other susceptible viruses. Successful development of constructs that can selectively silence specific viral promoters justifies the development of prophylactic derivatives for medical and veterinary applications and are also included as an embodiment of the instant invention.

4. Applications of Targeted Methylation to Other Gene Inactivation Studies

The methylating agents described here are very versatile, and can be targeted to other promoters by installation of zinc finger moieties of the appropriate sequence specificity. The choice of HIV-1 as a target in this proposal was motivated by the severe threat to public health posed by this virus rather than by any special vulnerability. Slight modification of the design and selection procedures can produce agents that methylate any predetermined sequence, and such agents may be useful in any case in medicine or experimental biology where it is desirable to silence a given promoter.

This invention is illustrated in the Experimental Detail section which follows. These sections are set forth to aid in an understanding of the invention but are not intended to, and should not be construed to, limit in any way the invention as set forth in the claims which follow thereafter.

Experimental Details

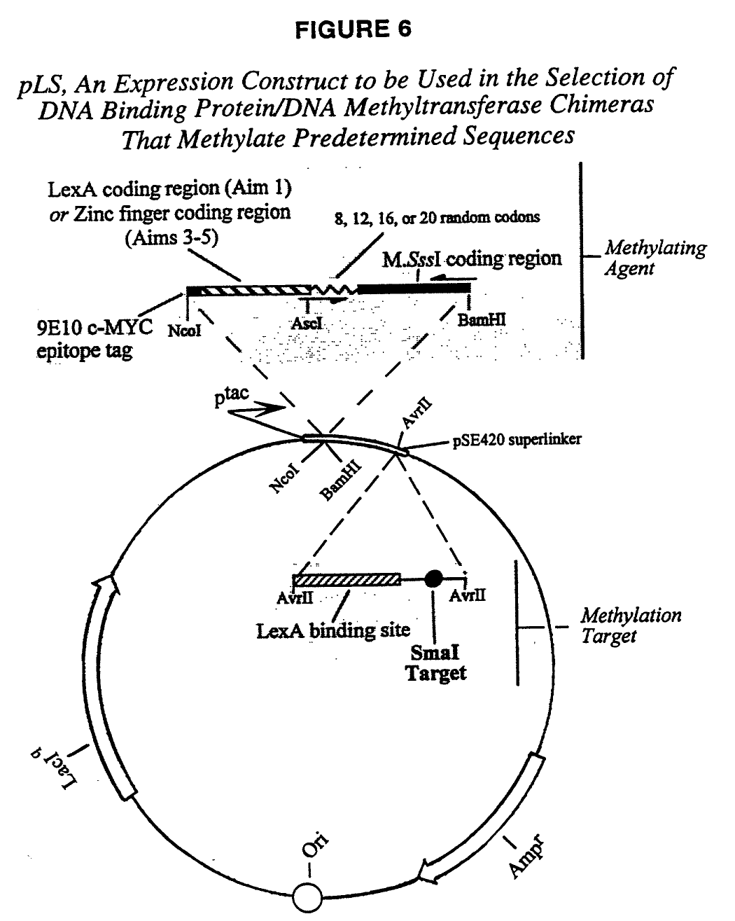

In the construction of a nucleic acid vector (see FIG. 6), LexA was chosen for its proven ability to direct fused proteins to LexA binding sites (Brent and Ptashne, 1986), and M.SssI was chosen because of its high specific activity and the fact that it is the only bacterial methyltransferase that has the same specificity (5′-CpG-3′; Renbaum and Razin, 1992) as the mammalian enzyme (Bestor and Ingram, 1983).

### EXAMPLE 1

Targeted de novo methylation.

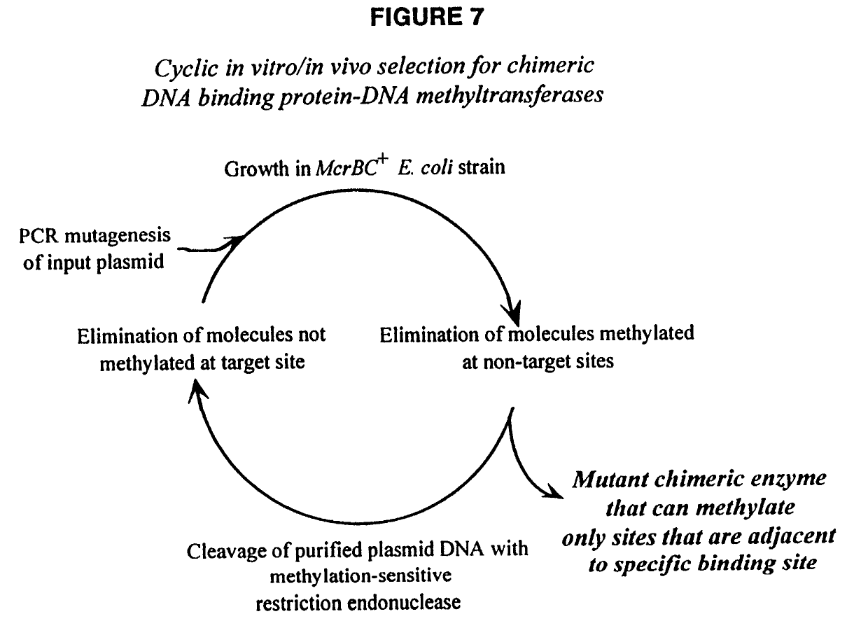

The product of the construct represented in FIG. 6 yields an increased local concentration of DNA methyltransferase in the vicinity of LexA binding sites. As described above, this alone does not ensure targeted methylation, as the M.SssI moiety retains intrinsic activity towards all CpG sites and substantial methylation of collateral sites is to be expected; such indiscriminate methylation is lethal to mammalian cells. It is therefore necessary to make the DNA methyltransferase moiety dependent on LexA-mediated DNA binding; this may be accomplished by selection of mutant versions of M.SssI that have reduced intrinsic DNA binding activity. A novel cyclic in vivo/in vitro selection protocol is used to select mutant proteins of the desired character. Resistance to cleavage by a methylation-sensitive restriction endonuclease provides in vitro selection for methylation of the target site; growth of cleavage-resistant pools of plasmid in McrBC⁺ strains of E. coli selects against chimeras that methylate non-target sites. (Mcr was named from modified cytosine restriction; the system causes the degradation of plasmid DNA that is methylated at many positions but has no effect on DNA that is methylated at single sites;Raleigh, 1992). The selection scheme is depicted in FIG. 7.

As mentioned above, the initial construct methylates both specific (that is, the SmaI target site) and non-specific CpG sites elsewhere on the plasmid. It is therefore necessary to transfer DNA binding authority from the catalytic moiety to the DNA binding moiety; this is done by selecting for mutations that prevent the methylation of non-specific sites while allowing methylation of the specific site.

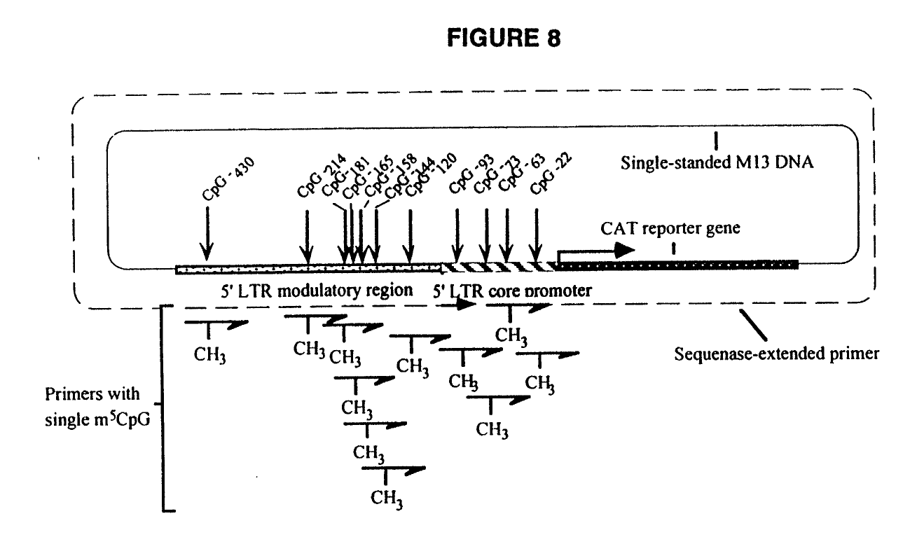

It cannot be predicted as to which mutations might give the desired reduction in affinity for DNA, so random mutations are introduced and selection is applied to obtain mutants of the desired character. The M.SssI moiety is mutagenized by Mn⁺⁺ PCR (PCR amplification in the presence of Mn⁺⁺ causes misincorporation of nucleotides and therefore transition and transversion mutations) from primers just external to the BamHI and AscI sites (the primers are depicted as arrows in FIG. 8). The PCR product is cleaved with BamHI and AscI, the fragment purified by gel electrophoresis and ligated between the AscI and BamHI sites of pLS (FIG. 6). The ligation products are electroporated into JM105 (McrBC⁻). Calculations indicate that optimal ligation and electroporation conditions allow selection to be applied to >10⁹ separate clones per screen (J. Hill and T. Bestor, data not shown). After 20 min growth in rich liquid medium at 37° C. and 150 min in rich medium supplemented with 50 μg ml⁻¹ ampicillin, the cells are washed 3 times and plasmid DNA extracted by the alkaline lysis procedure. The DNA is treated with 5 units μg⁻¹ SmaI for 2 hr at 25° C., then transformed into McrBC+ E. coli strain ER1381. As previously described, the McrBC system degrades DNA that is methylated at many sites but does not affect DNA methylated at single sites (Raleigh, 1992). It may be necessary to perform more than one iteration of the mutagenesis/selection procedure, but the process is not demanding and each iteration of the complete cyclic in vivo/in vitro selection method can be completed in a few days.

An additional selection step may be added to the cyclic selection procedure if the previous two steps give insufficient enrichment for the clones of interest. Plasmid pools are extracted from McrBC₌⁺ bacteria and cleaved with SmaI together with one of the 6 methylation-sensitive enzymes that have unique sites in pLS. If both sites are methylated, no cleavage occurs. If both sites are unmethylated, both will be cleaved. Only if one site is methylated and the other unmethylated will unit-length molecules with compatible ends be produced. These unit-length linear molecules are isolated by agarose gel electrophoresis, circularized by treatment with DNA ligase, and reintroduced into E. coli by electroporation. Note that this selection step, as in the case of the other two, can be completed in less than two days.

The procedure described above is designed to produce a chimeric protein that methylates a predetermined target site adjacent to the binding site of a sequence-specific DNA binding protein. Candidate clones may be confirmed by simple assays. To confirm that SmaI resistance is due to methylation of the target site and not to loss of the site by mutation, candidate plasmids are tested for resistance to SmaI and sensitivity to the methylation-insensitive isoschizomer XmaI (both enzymes recognize the sequence CCCGGG). Constructs that pass this test are examined for non-specific-methylation by testing for sensitivity to the methylation-sensitive endonucleases BstU1 (CGCG), HpaII (CCGG), MaeII (ACGT), HhaI (GCGC). Resistance and sensitivity is assessed from banding patterns after agarose gel electrophoresis. It can be concluded that targeted methylation was achieved upon recovery of plasmids that are resistant to SmaI and sensitive to XmaI, BstU1, HhaI, HpaII, and MaeII.

If clones of the desired binding and enzymatic specificity do not emerge from the screen, a likely reason may be stearic incompatibility of the DNA binding protein and DNA methyltransferase in the orientation shown in FIG. 6. New constructs may be made in which the order of the moieties are reversed, and the selection screen described above may be done on these new constructs.

The number of iterations of the mutagenesis/selection protocol that is required to produce constructs with the required characteristics may be variable. However, the procedure is rapid, and multiple iterations are not demanding.

Constructs that have the desired binding and enzymatic specificity are sequenced in order to identify mutations that make DNA methyltransferase activity dependent on the binding energy of the DNA binding fusion partner. The nature and position of the mutations are referred to the 3D structure of cytosine methyltransferases M.HhaI (Klimasauskas et al., 1994) and M.HaeIII (Reinisch et al., 1995); both of these enzymes are closely related to each other and to M.SssI, and conservation of tertiary structure is very likely. The desired mutations may map to one of the many residues that make nonspecific contacts with the DNA backbone (reviewed by Bestor and Verdine, 1994). These mutations are introduced into the starting constructs of Example 3 so as to reduce the amount of selection to obtain constructs of the desired character.

### EXAMPLE 2

Identification of those CpG sites in the HIV-1 5′ LTR whose methylation produces maximal repression of transcription.

As described previously, viral transcription is strongly repressed by methylation of the HIV-1 5′ LTR (reviewed by Bednarik, 1992). It is very likely that this effect is exerted through a subset of the 11 CpG sites present in the 5′ LTR of HIV-1 (see FIG. 1). The critical CpG sites are identified by measurements of transcription rates of test constructs that bear single methylated CpG sites. The CpG site that gives the highest degree of inhibition when methylated serves as the target for Examples 3 and 4.

The approach is related to methods established by Busslinger et al. (1983). The PstI-BamHI insert of pUC-BENN-CAT (which contains the HIV-1 5′ LTR driving transcription of the bacterial chloramphenicol acetyltransferase (CAT) gene) (Gendelman et al., 1986) is excised and cloned into M13 mp18. Single-stranded phage DNA is prepared, and synthetic oligonucleotide primers that bear m⁵C at single specific CpG sites are hybridized to an M13 clone of the sequence of interest. The primer is extended with a DNA polymerase; sequenase is the polymerase of choice for this invention because of its high processivity and low yield of partially extended products. The nick at the 5′ end of the primer is sealed by incubation with E. coli DNA ligase and the circular double stranded molecules are purified away from single stranded template and oligonucleotide primer by agarose gel electrophoresis. The circular DNA is purified from gel slices, cleared of ethidium bromide by extraction with phenol, collected by ethanol precipitation, and mixed with equimolar amounts of an actin promoter-luciferase expression construct prior to transfection into HL2/3 cells (Ciminale et al., 1990). This cell line expresses high levels of the HIV-1 proteins Gag, Env, Tat, Rev, and Nef proteins and affords a good simulation of HIV-1 transcription in infected T cells; it is especially important to identify CpG sites that suppress transcription when methylated and in the presence of the virus-encoded stimulatory factor Tat. The hemimethylated sites introduced into the constructs are converted to symmetrically methylated sites immediately upon transfection (Busslinger et al., 1983). The ratio of CAT activity to luciferase activity (both are measured by simple and well-established assays) are taken as the measure of inhibition by CpG methylation. Control constructs are either completely unmethylated or completely methylated (by extension of the primer in the presence of dm⁵CTP). The location of the 11 CpG sites and the method of synthesis of the methylated expression constructs is shown in FIG. 5. The plasmid pUC-BENN-CAT and the cell line HL2/3 are distributed by the NIH AIDS Research and Reference Reagent Program, and have been obtained from that source. This experiment is straightforward, and the results are invaluable for the experiments described in Examples 3, 4, and 5. The assays are done both in transiently transfected cells, and in cell clones that have stably integrated the construct; these clones are established by G418 selection after unlinked co-transformation with pSV2Neo and the pUC-BENN-CAT constructs.

### EXAMPLE 3

Design, selection, and affinity maturation of zinc finger-DNA methyltransferase chimeras that methylate critical CpG sites in the HIV 5′ LTR.

There are three important factors to be taken into account when selecting a site for targeted methylation in the HIV-1 5′ LTR. First, the site must have the ability to repress transcription when methylated; this is established in the experiments of Example 2. Second, the binding site for the zinc finger moiety must be adjacent to (rather than within) the recognition sequence of a regulatory factor. The factors that bind to the 5′ LTR are all host-encoded and are involved in the transcription of many cellular genes; methylation of such sites is expected to cause unacceptable toxicity through inactivation of vital cellular genes. Third, the recognition sequence is preferably from 6 to 25 base pairs in length and more preferably at least 9 base pairs in length, because such sequences occur by chance only every 4⁹ (or 262,144) base pairs. This rarity ensures that target sites do not occur by chance in significant numbers of cellular promoters.

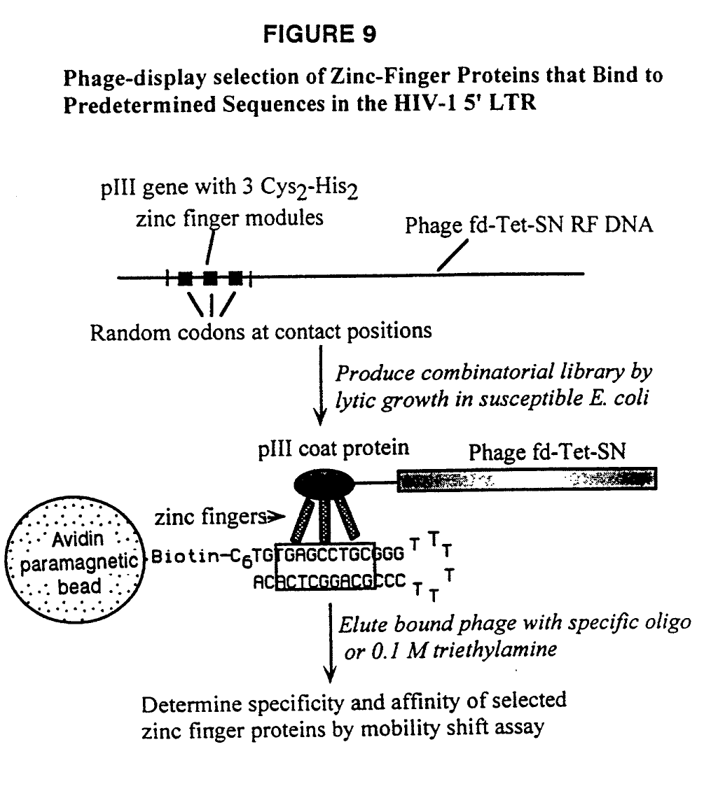

Zinc finger proteins that meet the above criteria are selected from combinatorial expression libraries by phage display, and fused to the CpG-specific DNA methyltransferase M.SssI as described in Example 1, and depicted in FIG. 9. Rounds of cyclic in vivo/in vitro selection (also described in Example 1) are used to obtain variants that methylate only CpG sites adjacent to the binding site of the zinc finger moiety. As before, more than one iteration of the mutagenesis/selection procedure may be required, but the process has no difficult or time-consuming steps. Only a few days are required for each iteration, and large numbers of clones (>10⁹ per cycle) can be subjected to selection at once.

The method for the selection of tridactyl zinc finger proteins that bind to predetermined sequences is shown in FIG. 9.

The method depicted in FIG. 9 is used to select a tridactyl finger protein that binds to a 9 bp sequence adjacent to a critical CpG site (identified in Example 2). The spacing between the recognition sequence and the target CpG site may be adjusted to conform to the sum of the estimated radii of the two globular proteins of masses 30,000 (M.SssI) and 20,000 (the tridactyl zinc finger protein). This spacing is equivalent to roughly 25 base pairs.

The selected finger protein is fused to M.SssI methyltransferase via a random linker to create a second combinatorial expression library. From this point forward the protocol is very similar to that described in Example 1. The construct differs from that shown in FIG. 6 only in that LexA is replaced with the coding region for the zinc finger protein, and the LexA binding site is replaced with the HIV-1 LTR sequence used for selection. The c-MYC epitope tag is included to allow visualization of the protein on immunoblots and by immunofluorescence. The sequence and spacing of the central CpG in the SmaI site and the recognition sequence for the zinc finger protein is the same as that in the HIV-1 LTR target sequence; a minimal change that converts the sequences flanking the CpG site to a SmaI site is introduced. This change should not pose any problems, as the intrinsic sequence specificity of M.SssI is limited to the CpG dinucleotide (Renbaum and Razin, 1993). As was previously done with the LexA construct, cyclic in vivo/in vitro selection is used to obtain mutant chimeras in which the DNA methyltransferase moiety depends on the finger protein part of the energy of DNA binding. This obviates methylation of collateral CpG sites, which is known to be toxic to mammalian cells. As with the LexA construct of Example 1, it may be confirmed that SmaI resistance is due to methylation of the target site, and not to loss of the site by mutation, by testing for resistance to SmaI and sensitivity to the methylation-insensitive isoschizomer XmaI. Constructs that pass this test are examined for non-specific methylation by testing for sensitivity to the methylation-sensitive endonuclease BstU1 (CGCG), HpaII (CCGG), MaeII (ACGT), HhaI (GCGC). Almost 100 CpG sites within the plasmid construct may be tested through use of these enzymes. Resistance and sensitivity is assessed from banding patterns after endonuclease treatment and agarose gel electrophoresis. As in Example 1, it may be concluded that targeted methylation has been achieved upon recovery of plasmids that are resistant to cleavage by SmaI and sensitive to XmaI, BstUl, HhaI, HpaII, and MaeII.

Alternatively, as with the LexA construct, constructs in which the order of the zinc finger and M.SssI moieties has been reversed are constructed and subjected to the selection protocol described above. As in the case of the pLS constructs described in Example 1, the data indicate that it is possible to screen between 10⁹and 10¹⁰ independent clones at once, and each iteration of the selection protocol requires only a few days.

### EXAMPLE 4

Inhibition of HIV-1 5′ LTR-dependent transcription in cultured human cells that express zinc finger/DNA methyltransferase fusion proteins of novel and predetermined specificity.

Example 3 yields constructs that methylate critical sites in the HIV-1 5′ LTR. These constructs are introduced into cultured human cells that express a reporter gene from the HIV-1 5′ LTR as a means of testing the inhibitory effects of targeted de novo methylation.

The plasmid pUC-BENN-CAT (Gendelman et al., 1986) is introduced into HL2/3 cells (Ciminale, 1990) together with unlinked pSV2Neo, and stable transfectants selected with G418. The HL2/3 cell line expresses most of the HIV-1 accessory proteins (including Tat) from a replication-defective HIV-1 provirus (Ciminale et al., 1990). The presence of HIV-1 viral accessory proteins renders this assay a good simulation of HIV-1 transcription in infected cells. Transfectant clones that express the CAT gene from the HIV-1 LTR are identified by RNA blot hybridization. Such cell clones are referred to as HL2/3-LTR-CAT.

HL2/3-LTR-CAT cells are transfected by the calcium phosphate technique with a mammalian expression vector that directs production of the zinc finger-M.SssI fusion protein (Example 3) from the cytomegalovirus (CMV) immediate early enhancer-promoter. The starting vector is pEVRFO (Matthias et al., 1990), which contains the CMV promoter, a beta globin intron in the 3′ untranslated region for enhanced transcript stability, and a polylinker region for convenient cloning. We have used this vector with good success in many constructs (Czank et al., 1991; Leonhardt et al., 1992). This expression construct is termed pCMV-ZMet. A pSV2Hyg resistance marker is co-transfected with pCMV-ZMet, and the cells subjected to double selection with G418 and Hygromycin. Production of zinc finger/M.SssI fusion protein are assayed by immunoblot analysis; as with the LexA-M.SssI fusion protein of FIG. 3, an N-terminal c-MYC epitope tag is present, and the protein is visualized with the 9E10 antibody.

Immunofluorescence microscopy is used to determine whether the methylating agent can gain access to nuclei. If it is found to be confined to the cytoplasm, the nuclear localization signal of SV40 large T antigen may be added to the N-terminus, as we have done for a class of fusion proteins to induce their translocation into nuclei (Leonhardt et al., 1992).

Cells that express zinc finger/M.SssI fusion protein are examined for loss of CAT expression by CAT enzyme assay and by RNA blot hybridization. In the event that the most critical CpG site happens to lie within one of the four sites for methylation-sensitive restriction endonuclease (four of the eleven sites can be assayed in this way; see FIG. 1), methylation may be assayed by cleavage of DNA with that enzyme followed by DNA blot hybridization. This is a simple and long-established technique for determination of the methylation status of individual CpG sites. If the critical site chosen for targeted methylation does not lie within such a restriction site, the genomic sequencing method of Grigg and Clark. (1994) may be used to identify methylated cytosines. The method of Collins and Myers (1987) can also be used to detect methylation differences. This method exploits the increased melting temperature of methylated DNA, which allows methylated and unmethylated fragments to be resolved by denaturing gradient gel electrophoresis (Collins and Myers, 1987). Both methods are well-established and both will allow unambiguous localization of methylated sites in HIV-1 LTR sequences.

The embodiments described herein of this invention indicate that expression of a finger protein/M.SsSI chimera targeted to a critical CpG site in the HIV-1 LTR methylates and inactivates expression of a reporter gene that is driven by the LTR.

### EXAMPLE 5

Inhibition of HIV-1 replication in T lymphocytes productively infected with EIV-1.

Jurkat E6-l cells are electroporated with infectious molecular clones of HIV-1 strain NL4-3, and a productive infection established. The HIV-producing cells are transfected with the finger protein/M.SssI construct described in the previous section, together with pSV2Neo as a selectable marker. A vector control and a finger protein-only control may be used. This latter control is necessary to distinguish inhibition via methylation from that resulting from direct binding of the finger protein to its target site, as has happened in one other case (Choo et al., 1995). The finger protein/M.SssI chimera is designed to cause de novo methylation and transcriptional repression of HIV-1 proviral DNA. Methylation is assayed as described in the previous section, and viral transcription is assayed by RNA blot hybridization.

The plasmid-mediated system test described above is convenient and reliable but does not simulate conditions that may be encountered in a therapeutic application. A vector that addresses the needs of therapeutic applications is therefore used and described below. One of the most promising viral vectors is based on adeno-associated virus (AAV), and the methylating agent may be delivered through use of recombinant AAV.

AAV has several advantages as a transducing vector: I. AAV is not associated with any disease, ii. The viral DNA is thought to integrate at a unique site on chromosome 19q, with little risk of insertional mutagenesis, iii. it has been reported that AAV can infect non-replicating cells (reviewed in Berns and Linden, 1995), iv. AAV can infect lymphoid cells (Mendelson et al., 1992), and transcription units borne by recombinant AAV strains are expressed in T lymphocytes (Chatterjee et al., 1992), and v. Virulent strains of AAV cannot arise by recombination within infected cells, as can occur with retroviral vectors.

Introduction of methylating agents into transducing AAV vectors is straightforward. The finger protein/DNA methyltransferase coding region under control of the CMV immediate early enhancer-promoter is amplified by PCR with primers that introduce XbaI sites at both ends of the construct. This expression cassette is ligated between the XbaI sites of pAV1, an infectious molecular clone of AAV (Banerjee et al., 1992). The resulting clones are selected and propagated in E. coli, and confirmed by direct sequencing; clones of the desired specificity are termed pAV-ZMet. Control clones that express only the zinc finger protein are constructed and termed pAV-Zif; clones that express only the mutant M.SssI are constructed and termed pAV-Met. Infectious recombinant virus stocks are obtained by co-transfection of adenovirus-infected HeLa cells with pAV-ZMet, pAV-Zif, or pAV-Met, together with pTAAV, a plasmid that encodes proteins required for replication and packaging (Banerjee et al., 1992). The titer of virus stocks obtained in this manner are commonly 10⁷ ml¹ (Banerjee et al., 1992); such viruses are also capable of infecting CD4⁺ T lymphocytes such as Jurkat cell with high efficiency (Mendelson et al., 1992). The recombinant viruses are termed AAV-ZMet, AAV-Zif, and AAV-Met, in keeping with the terminology of their plasmid precursors.

Jurkat cells productively infected with HIV-1 strain NL4-3 (as described above) are infected with AAV-ZMet, AAV-Zif, or AAV-Met at an MOI that infects >90% of the cell population, as established by immunofluorescence microscopy with antibodies against the c-MYC epitope encoded by the fusion proteins. Jurkat cells are highly susceptible to infection by AAV (Mendelson et al., 1992) RNA blot hybridization is used to evaluate the effect of expression on transcript accumulation, as has been done in antisense inhibition experiments that utilized AAV expression vectors (Banerjee et al., 1992). The methylation status of integrated HIV-1 proviral DNA may be evaluated as described in Example 3. Comparison of transcript levels, cell survival, and proviral methylation among cultures infected with vector control, AAV-ZMet, AAV-Zif, and AAV-Met can give a clear indication of the efficacy of targeted methylation in suppressing HIV-1 expression under conditions that approximate those to be encountered in actual therapeutic applications.

### EXAMPLE 6

Design, selection, and affinity Maturation of zinc finger-DNA methyltransferase chimeras that methylate critical CpG sites in a promoter of the hepatitis B virus.

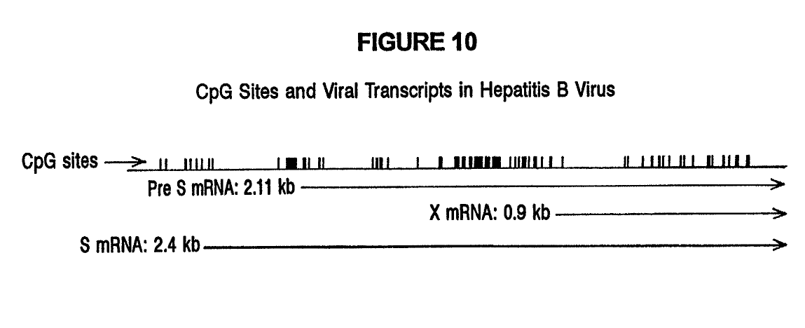

Chronic hepatitis due to infection with Hepatitis B virus (HBV) is a major cause of cirrhosis of the liver and hepatocellular carcinoma (reviewed by Ganem and Varmus, 1987). Approximately 1.5% of the population is infected with HBV (reviewed by Saracco and Rezzetto, 1995) and the worldwide morbidity and mortality associated with chronic HBV infection is far greater than that due to HIV-1 infection. As shown in FIG. 10, the HBV genome is rich in CpG sites (there are 103 in a genome of only 3.2 kb; Ono et al., 1983) and transcription from HBV promoters is sensitive to CpG methylation (Miller and Robinson, 1983; Pourcel et al., 1990; The contents of these references are incorporated in their entirety into the subject application). HBV proviral DNA is therefore susceptible to inactivation by targeted methylation according to the methods elaborated for HIV-1 inactivation herein.

Identification of critical CpG sites, design of chimeric zinc finger protein/DNA methyltransferases, and cyclic in vivo/in vitro selection of chimeras that methylate only the target CpG site is as described for the HIV-1 targets in the above examples.

One embodiment of the subject invention is to target the known promoters of the hepatitis B virus and inactivate the viral machinery via methylation. The site for targeted methylation in the hepatitis B promoter is selected as follows. First, the site will have the ability to repress transcription when methylated; this is established in the experiments of Example 2 above. Second, the binding site for the zinc finger moiety will be adjacent to (rather than within) the recognition sequence of a regulatory factor. The factors that bind to the promoter of the viral X protein or the pre-S2/5 promoter are host-encoded and are involved in the transcription of many cellular genes; methylation of such sites is expected to cause unacceptable toxicity through inactivation of vital cellular genes. Third, the recognition sequence is optimally from 6 to 25 base pairs in length and more preferably at least 9 base pairs in length.

Zinc finger proteins that meet the above criteria are selected from combinatorial expression libraries by phage display, and fused to the CpG-specific DNA methyltransferase M. SssI as described in Example 1, and depicted in FIG. 9. Rounds of cyclic in vivo/in vitro selection (also described in Example 1) are used to obtain variants that methylate only CpG sites adjacent to the binding site of the zinc finger moiety. As before, more than one iteration of the mutagenesis/selection procedure may be required, but the process has no difficult or time-consuming steps. Only a few days are required for each iteration, and large numbers of clones (>10⁹ per cycle) can be subjected to selection at once.

The method for the selection of tridactyl zinc finger proteins that bind to predetermined sequences is shown in FIG. 9.

The method depicted in FIG. 9 is used to select a tridactyl finger protein that binds to a 9 bp sequence adjacent to a critical CpG site in either the promoter of viral protein X or the pre-S2/5 promoter of hepatitis B. The spacing between the recognition sequence and the target CpG site may be adjusted to conform to the sum of the estimated radii of the two globular proteins of masses 30,000 (M.SssI) and 20,000 (the tridactyl zinc finger protein). This spacing is equivalent to roughly 25 base pairs.

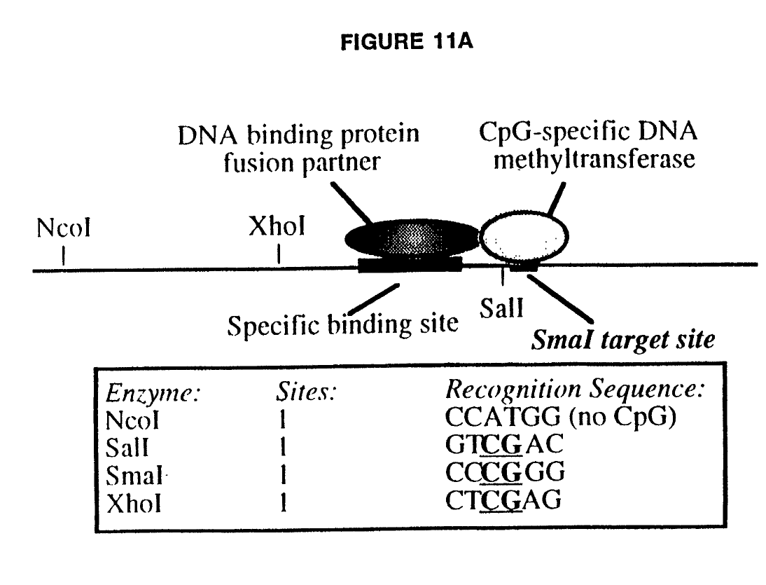

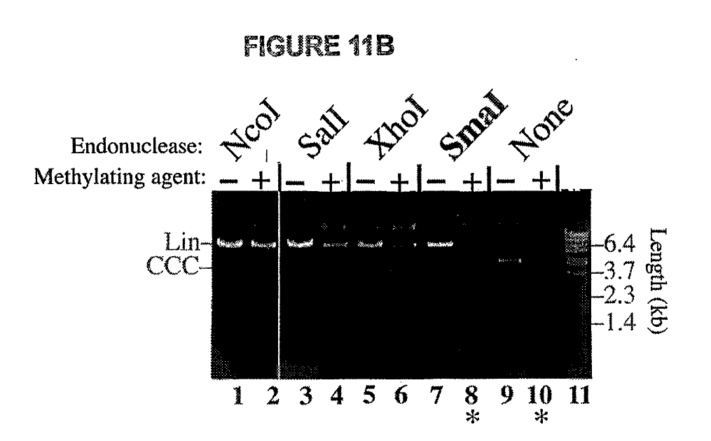

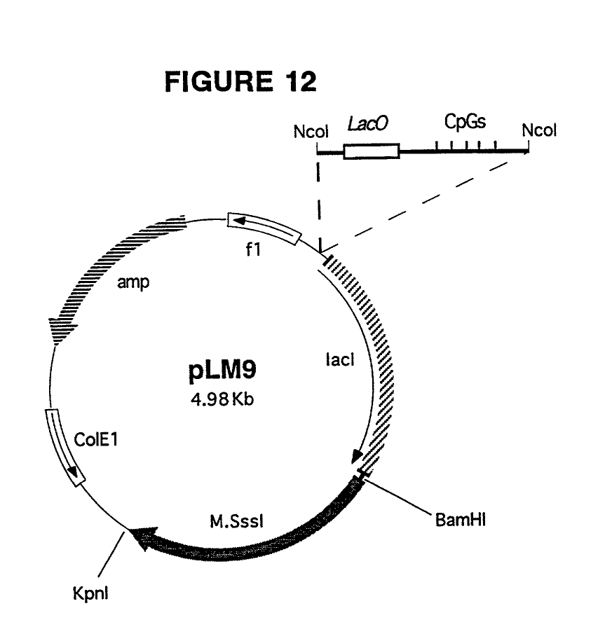

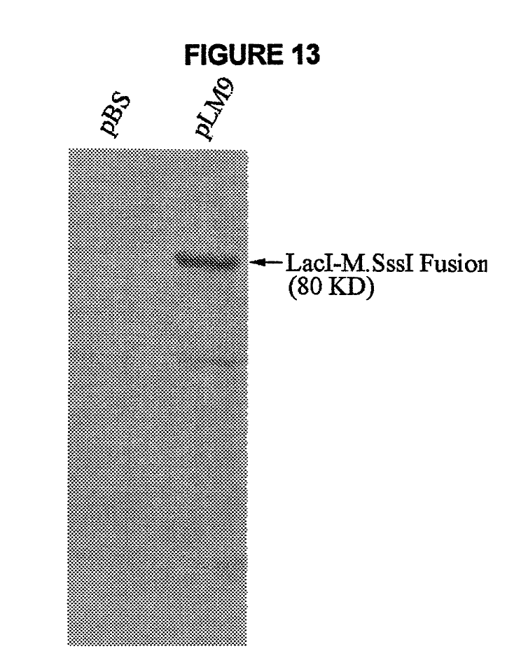

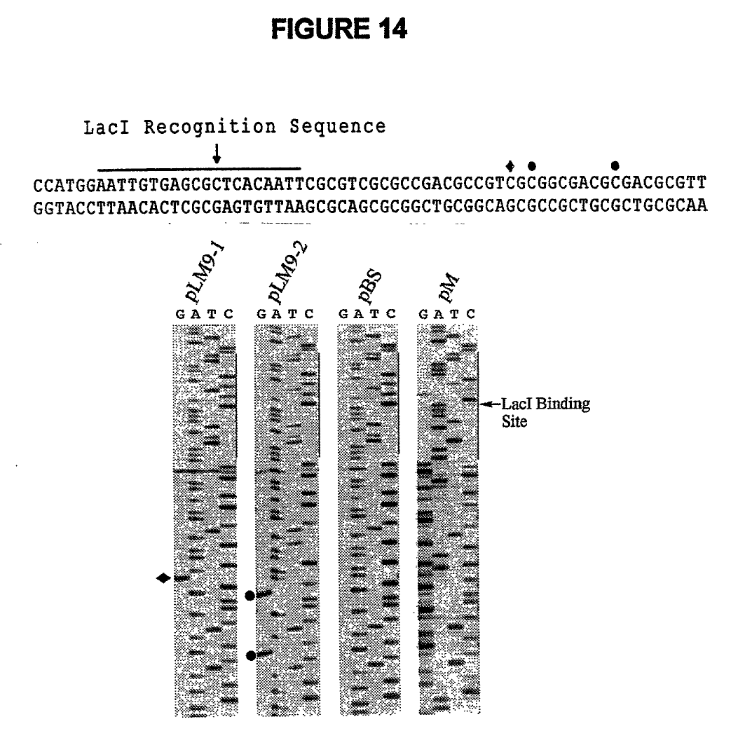

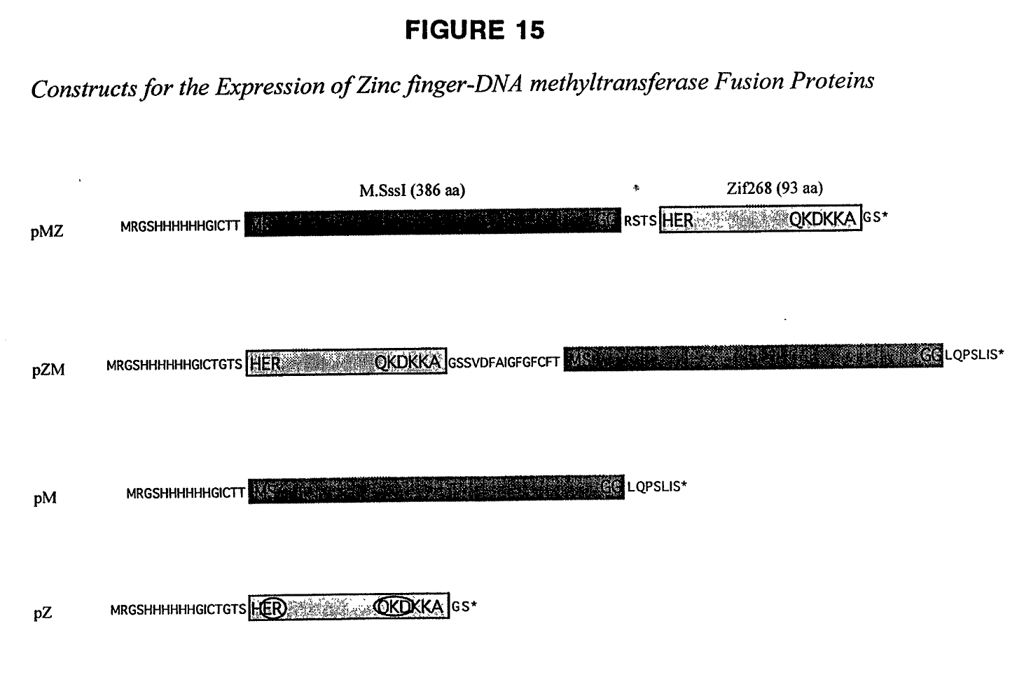

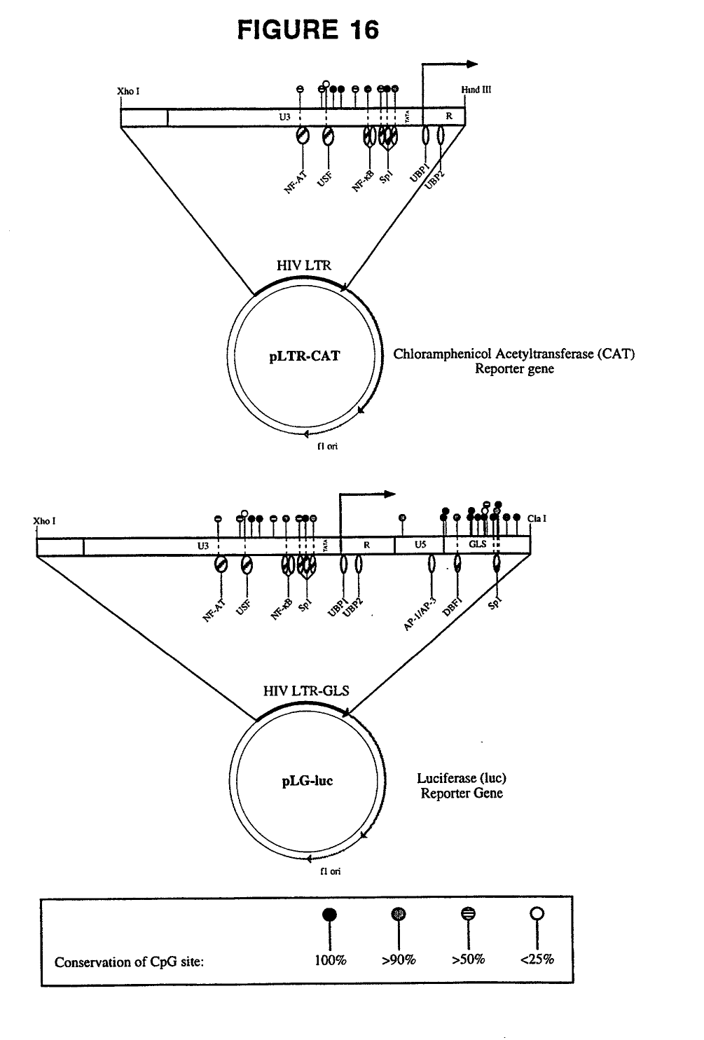

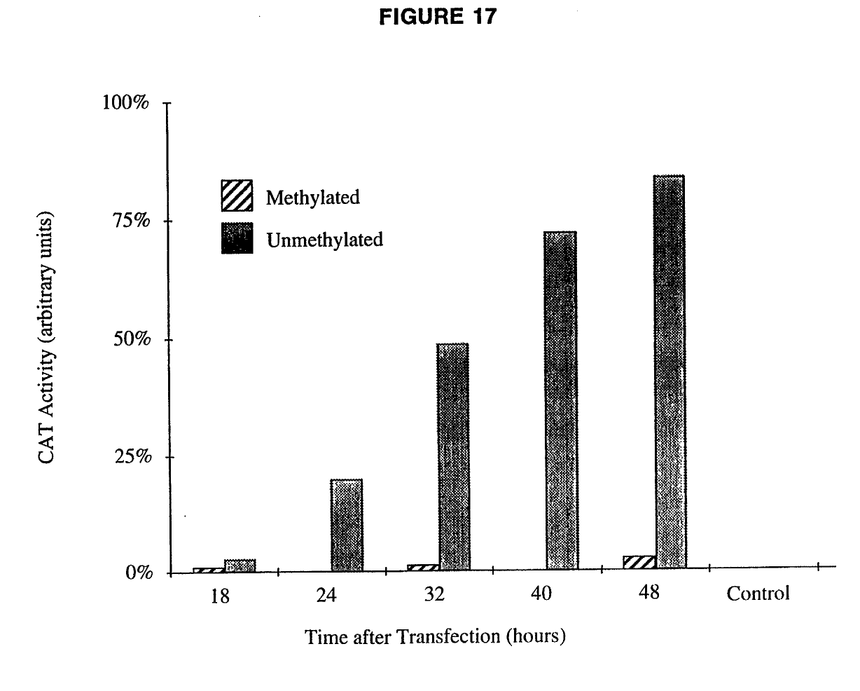

FIG. 11 A and FIG. 11B illustrate targeted methylation of a preselected CpG site in a pLS vector which encodes a lexA-M.SssI fusion protein. This data proves that a CpG-specific DNA methyltransferase can be directed to a target site that is adjacent to the binding site of a sequence-specific DNA binding protein. This targeted methylation was achieved in living bacterial cells. Mutagenesis and cyclic in vivo/in vitro selection is being applied so as to obtain a chimeric protein that will methylate the target site but have no effect on collateral CpG sites. The data shown above and specifically in FIGS. 11A-11B, demonstrate that the principle of targeted methylation has been reduced to practice in the laboratory.

### EXAMPLE 7

Targeted methylation in the repression of HIV-1 replication

A. Construction of chimeric DNA methyltransferase of novel and predetermined specificity.

The Lac repressor was fused to the N-terminus of M.SssI by recombinant DNA technology and the chimeric protein was expressed in E. coli. The construct used in these experiments is shown in detail in FIG. 12. The construct also contained a synthetic sequence that included the Lac repressor binding site and a cluster of CpG dinucleotides. This construct was used to establish three important points: First, that chimeric DNA methyltransferase could be produced in bacterial cells in a stable and soluble form; second, that the chimeric DNA methyltransferase would selectively methylate CpG sites in the vicinity of the recognition sequence of the lac repressor, and third, the optimal spacing between recognition sequence and target CpG site is 20-30 nucleotides.

The construct shown in FIG. 12 was electroporated into E. coli and synthesis of fusion protein induced by addition of isopropylthiogalactoside to culture medium. After 3 hours of incubation cells were lysed, and protein extracts were subjected to SDS gel electrophoresis and immunoblot analysis with an anti-Lac repressor antibody. As shown in FIG. 13, the chimeric DNA methyltransferase was soluble and resistant to proteolysis as shown by its appearance as a single band of the appropriate size. There was little sign of a reduction in growth rate upon induction of synthesis of the chimeric DNA methyltransferase. This confirmed that the chimeric DNA methyltransferase was not toxic to the host cells.

Plasmid DNA was purified from the same cells and high resolution mapping of methylated sites was carried out by the bisulfate genomic sequencing method. Bisulfite converts cytosine to uracil by oxidative deamination. 5-methylcytosine is resistant to bisulfite attack. Upon PCR amplification all cytosine will be copied as thymidines but 5-methylcytosine will be preserved as cytosines. As shown in FIG. 14, plasmid DNA exposed in vivo to Lac repressor-M.SssI fusion proteins underwent methylation at CpG sites near the Lac repressor binding site; vector DNA was not methylated, and plasmid exposed to M.SssI became methylated at all CpG sites. These data confirm that fusing a DNA methyltransferase to a sequence-specific DNA binding protein can limit methylation to CpG sites in the vicinity of the recognition sequence of the DNA binding protein as originally proposed.

FIG. 14 also shows that targeted methylation occurred 20-30 nucleotides away from the 3′ border of the Lac repressor binding site. This is taken to reflect steric interference between protein moieties. The result was expected and is not a limitation of the method.

The Lac repressor-M.SssI fusion proteins confirmed the principle of targeted methylation. FIG. 15 depicts a new class of chimeric DNA methyltransferases that have been constructed and confirmed by DNA sequencing. The sequence-specific DNA binding moiety is a three-finger zinc finger protein (Zif268) of known binding specificity. These agents are being subjected to the same characterization as the Lac repressor-M.SssI fusions. They have also been cloned into mammalian expression vectors and experiments in cultured primate cells are being performed.

In summary, chimeric DNA methyltransferase-DNA binding proteins have been shown to selectively methylated CpG sites in the vicinity of the recognition sequence of the DNA binding protein moiety.

B. Inactivation of HIV-1 by CpG methylation.

As shown in FIG. 16, the LTR of HIV-1 has been cloned upstream of two reporter genes, chloramphenicol acetyltransferase (CAT) and firefly luciferase (Luc). Both constructs drive high-level expression of the CAT of Luc reporter genes when transfected into tat-expressing HL2/3 or HLTAT human cervical carcinoma cells.

Methylation is known to suppress transcription from most promoters but its effect on HIV-1 LTR-driven transcription has not been documented in a system that resembles the infected cells. FIG. 17 shows that CpG methylation of 9 CpG sites in the HIV-1 LTR reduces transcription to near background levels; the effect is at least 20 fold. The effect appears to be irreversible and does not require continued application or expression of any foreign agents.

CpG methylation can therefore be regarded as the most powerful way to suppress the activity of HIV-1. Work is underway that will identify the CpG sites that mediate the biological effects of methylation.

Work as described herein, has shown that M.SssI does not methylate single stranded DNA. This has led to the development of a new method for the region-specific methylation of a DNA fragment. A single-stranded oligonucleotide or longer fragment synthesized by asymmetric PCR is annealed to a single-stranded circular DNA and treated with M.SssI. CpG sites in the double stranded region of the plasmid are methylated while all single stranded regions are unmodified. This new method reduces the time required to identify critical CpG sites. The regional methylation method is being used to evaluate the effects of CpG methylation on the HIV-1 LTR versus the gag leader sequence; this latter sequence is rich and has strongly conserved CpG sites (FIG. 16) and methylation of the sites may suppress transcription of HIV-l as well or better than methylation of LTR sequences.

References

Ashburner, M. Drosophila: A Laboratory Handbook. Cold Spring Harbor Laboratory Press, Cold Spring Harbor, New York, 1992. p303.

Baltimore, D. (1995) Overview: the enigma of HIV infection. Cell 82, 175-177.

Bednarik, D. P. (1993) DNA methylation and retrovirus expression. EXS 64, 300-329

Bednarik, D. P. (1992) in DNA Methylation, J.-P. Jost and H.-P. Saluz, eds. Birkhauser, Geneva

Bednarik, D., J. Cook, and P. M. Pitha (1990). Inactivation of the HIV LTR by DNA CpG methylation: evidence for a role in latency. EMBO J. 9, 1157-1164.

Berns, K. I., and R. M. Linden (1995). The cryptic life style of adeno-associated virus. Bioessays 17, 237-245.

Bestor, T. H. (1987). Supercoiling-dependent sequence specificity of mammalian DNA methyltransferase. Nucl. Acids Res. 15, 3835-3843

Bestor, T. H. (1992). Activation of mammalian DNA methyltransferase by cleavage of a Zn-binding regulatory domain. EMBO J.11, 2611-2618

Bestor, T. H. (1990). DNA methylation: How a bacterial immune function has evolved into a regulator of gene expression and genome structure in higher eukaryotes. Phil. Trans. Royal Soc. Lond. B 326, 179-187

Bestor, T. H., and Coxon, A. (1993). The pros and cons of DNA methylation. Curr. Biol. 3, 384-386.

Bestor, T. H., and Ingram, V. (1985). Growth-dependent expression of multiple species of DNA methyltransferase in murine erythroleukemia cells. Proc. Nat. Acad. Sci. USA 82, 2674-2678

Bestor, T. H., and Ingram, V. (1983). Two species of DNA methyltransferase from murine erythroleukemia cells. Purification, sequence specificity, and mode of interaction with DNA. Proc. Nat. Acad. Sci. USA 80, 5559-5563

Bestor, T. H., and Verdine, G. L. (1994). DNA methyltransferases. Curr. Op. Cell Biol. 6, 380-389.

Bestor, T. H., Laudano, A., Mattaliano, R., and Ingram, V. (1988). Cloning and sequencing of a cDNA encoding DNA methyltransferase of mouse cells. The carboxyl-terminal domain of the mammalian enzyme is related to bacterial restriction methyltransferases. J. Mol. Biol. 203, 971-983

Bowerman, B., P. O. Brown, J. M. Bishop, and H. E. Varmus (1989). A nucleoprotein complex mediates the integration of retroviral DNA. Genes Devel. 3, 469-478.

Brent, R., and Ptashne, M. (1985) A eukaryotic transcriptional activator bearing the DNA specificity of a prokaryotic repressor. Cell 43, 729-736

Bushman, F. D. (1994) Tethering human immunodeficiency virus I integrase to a DNA site directs integration to nearby sequences. Proc. Nat. Acad. Sci USA 91, 9233-9237.

Busslinger, M., J. Hurst, R. A. Flavell (1983) DNA methylation and the regulation of globin gene expression. Cell 34,197-206

Carlson, L. L., Page, A. W., and Bestor, T. H. (1992). Localization and properties of DNA methyltransferase in preimplantation mouse embryos: Implications for genomic imprinting. Genes & Development 6, 2536-2541

Cassens, S., and U. Ulrich (1994). Inhibition of human T cell leukemia virus type I long terminal repeat expression by DNA methylation: implications for latency. J. Gen. Virol. 75, 3255-3259.

Challita, P.-A., and D. B. Kohn (1994). Lack of expression from a retroviral vector after transduction of murine hematopoietic stem cells is associated with methylation in vivo. Proc. Nat. Acad. Sci. USA 91, 2567-2571.

Chatterjee, S., P. R. Johnson, K. K. Wong (1992). Dual-target inhibition of HIV-1 in vitro by means of an adeno-associated virus antisense vector. Science 258, 1485-1488.

Choo, Y., and A. Klug (1994). Toward a code for the interactions of zinc fingers with DNA: selection of randomized fingers displayed on phage. Proc. Nat. Acad. Sci. USA 91, 11163-11167.

Choo, Y., I. Sanchez-Garcia, and A. Klug (1994). In vivo repression by a site-specific DNA-binding protein designed against an oncogenic sequence. Nature (London) 372, 642-646.

Ciminale, V., B. K. Felber, M. Campbell, and G. N. Pavlakis (1990). A bioassay for HIV-1 based on Env-CD4 interaction. AIDS Res. 6, 1281-1287.

Collins, M, and R. M. Myers (1987) Alterations in DNA helix stability due to base modifications can be evaluated using denaturing gradient gel electrophoresis. J. Mol. Biol.198, 737-744

Czank, A., Hergersberg, M., Hauselman, R., Page, A., Leonhardt, H., Bestor, T. H., and Schaffner, W. (1991). Expression of the gene for mammalian DNA methyltransferase in mammalian cells. Gene 109, 259-263

Desjarlais, J. R., and J. M. Berg (1993). Use of a zinc-finger consensus sequence framework and specificity rules to design specific DNA binding proteins. Proc. Nat. Acad. Sci. USA 90, 2256-2260.

Desjarlais, J. R., and J. M. Berg (1992) Redesigning the DNA-binding specificity of a zinc finger protein: a database-guided approach. Proteins 12, 101-104.

de Fagagna, F., G. Marzio, M. I. Gutierrex, L. Y. Kang, A. Falaschi, and M. Giacca (1995). Molecular and functional interactions of transcription factor USF with the long terminal repeat of human immunodeficiency 1. J. Virol. 69, 2765-2775.

Ganem, D., and H. E. Varmus (1987) The molecular biology of the hepatitis B virus. Ann. Rev. Biochem. 56, 651-693.

Garcia, J. A., and R. B. Gaynor (1994) The human immunodeficiency virus type-1 long terminal repeat and its role in gene expression. Prog. Nucl. Acid Res. Mol. Biol. 49, 157-195.

Gendelman, H. E., W. Phelps, L. Feigenbaum, J. M. Ostrove, A. Adachi, P. M. Howley, G. Khoury, H. S. Ginsberg, and M. A. Martin (1986). Transactivation of the Human Immunodeficiency virus long terminal repeat by DNA viruses. Proc. Nat. Acad. Sci. USA 83, 9759-9763.

Grigg, G., and S. Clark (1994) Sequencing 5-methylcytosine residues in genomic DNA. Bioessays 16, 431-436

Holliday, R. (1993) Epigenetic inheritance based on DNA methylation. EXS 64, 452-68

Herman, J. G., F. Latif, Y. Weng, M. I. Lerman, B. Zbar, S. Liu, D. Samid, D.-S. Duan, J. R. Gnarra, W. M. Linehan, and S. B. Baylin (1994) Silencing of the VHL tumor suppressor gene by DNA methylation. Proc. Nat. Acad. Sci. USA 91, 9700-9704.

Joel, P., W. Shao, and K. Pratt (1993) A nuclear protein with enhanced binding to methylated Sp1 sites in the AIDS virus promoter. Nucl. Acids Res. 21, 5786-5793.

Jähner, D., and R. Jaenisch (1984). in DNA Methylation, H. Cedar and A. Riggs, Eds. Springer Verlag, New York.

Jaenisch, R., A. Schnieke, and K. Harbers (1985). Treatment of mice with 5-azacytidine efficiently activates silent retrovirus genomes in different tissues. Proc. Nat. Acad. Sci. USA 82, 1451-1455.

Krüger, T., C. Wild and M. Noyer-Weidner (1995). McrB: A prokaryotic protein specifically recognizing DNA containing modified cytosine residues. EMBO J. 14, 2661-2669.

Landau, N. R., and D. R. Littman (1992) Packaging system for rapid production of murine leukemia virus vectors with variable tropism. J. Virol. 66, 5110-5113

Leonhardt, H., and Bestor, T. H. (1992). Structure, function, and regulation of mammalian DNA methyltransferase. in DNA Methylation: Molecular Biology and Biological Significance. J.-P. Jost and H. Saluz, eds. Birkhauser, Geneva. pp 109-119.

Leonhardt, H., Page, A. W., Weier, H.-Ul., and Bestor, T. H. (1992). A targeting sequence directs DNA methyltransferase to sites of DNA replication in mammalian nuclei. Cell 71, 865-874.

Li, E., Beard, C., Forster, A. C., Bestor, T. H., and Jaenisch, R. (1993). DNA methylation, genomic imprinting, and mammalian development. Cold Spring Harb. Symp. Quant. Biol., LVIII, 297-305

Li, E., Bestor, T. H., and Jaenisch, R. (1992). Targeted mutation of the DNA methyltransferase gene results in embryonic lethality. Cell 69, 915-926

McCune, J. M. (1995). Viral latency in HIV disease. Cell 82, 183-188.

Meehan, R., J. Lewis, S. Cross, X. S. Nan, P. Jeppesen, and A. Bird. (1992) Transcriptional repression by methylation of CpG. J. Cell Sci. (suppl 16), 9-14

Mendelson, E., Z. Grossman, F. Mileguir, G. Rechavi, and B. J. Carter (1992). Replication of adeno-associated virus type 2 in human lymphocytic cells and interaction with HIV-1. Virology 187, 453-458.

Nabel, G. J. (1993). The role of cellular transcription factors in the regulation of human immunodeficiency virus gene expression. pp.49-73. in B. R. Cullen, ed., Human Retroviruses. IRL Press, Oxford.

Nardelli, J., T. Gibson, and P. Charnay. (1992) Zinc finger DNA recognition: analysis of base specificity by site-directed mutagenesis. Nucl. Acids Res. 20, 4137-4144.

Ono, Y., Onda, H., Sasada, R., Iarashi, K., Sugino, Y. And Nishioka, K. (1983) The complete nucleotide sequences of the cloned hepatitis B virus DNA. Nucleic Acids Res. 11, 1747-1757.

Pourcel, C., Tiollais, P., and Farza, H. (1990) Transcription of the S gene in transgenic mice is associated with hypomethylation at specific sites and with Dnase I sensitivity. J. Virol. 64, 931-935.

Prendergast, G. C., and E. B. Ziff (1991). Methylation-sensitive sequence-specific DNA binding by the c-Myc basic region. Science 251, 186-189.

Sarracco, G., and Rissetto, M. (1995). Recent results in the treatment of chronic B virus hepatitis. Biomed. Pharmacother. 49, 55-57.

Sutherland, E., L. Coe and E. A. Raleigh (1992). McrBC: A multisubunit GTP-dependent restriction endonuclease. J. Mol. Biol. 225, 327-348.

Raleigh, E. A. (1992) Organization and function of the mcrBC genes of Escherischia coli K-12. Mol. Micro. 6, 1079-1086.

Rebar, E. J., and C. O. Pabo (1994) Zinc finger phage: affinity selection of fingers with new DNA-binding specificities. Science 263, 671-673.

Reinisch, K. M., L. Chen, G. L. Verdine, and W. N. Lipscomb (1995). The crystal structure of HaeIII methyltransferase covalently complexed to DNA: An extra helical cytosine and rearranged base pairing. Cell 82, 143-153.

Renbaum, P., and A. Razin (1992) Mode of action of the Spiroplasma CpG methylase M.SssI. FEBS. Lett. 313, 243-247

Subbramanian, R. A., and E. A. Cohen (1994). Molecular biology f the human immunodeficiency virus accessory proteins. J. Virol. 68, 6831-6835.

Trasler, J., Alcivar, A., Hake, L., Bestor, T. H., and Hecht, N. (1992). Meiotic expression of a unique DNA methyltransferase transcript in the mouse male germ line. Nucleic Acids Res.20, 2541-2545

Volberding, P. (1995). The need for additional options in the treatment of human immunodeficiency virus infection. J. Infect. Des. 171, 150-154.

Wigler, M., Levy, D. and Perucho, M. (1981). The somatic inheritance of DNA methylation. Cell 24: 33-40.

Wu, H., W.-P. Yang, and C. F. Barbas (1995). Building zinc fingers by selection:Toward a therapeutic application. Proc. Nat. Acad. Sci. USA 92, 344-348.

Yoshiura, K., Y Kanai, A. Ochiai, Y. Shimoyama, T. Surimura, and S. Hirohashi (1995). Silencing of the E-cadherin invasion-suppressor gene by CpG methylation in human carcinomas. Proc. Nat. Acad. Sci. USA 92, 7416-7419.

What is claimed is:

1. A chimeric protein which comprises a mutated DNA methyltransferase portion and a DNA binding protein portion that binds sufficiently close to a promoter sequence of a target gene, which promoter sequence contains a methylation site, to specifically methylate the site and inhibit activity of the promoter and thus inhibit expression of the target gene.

2. The protein of claim 1, wherein the promoter sequence of the target gene is a 5′ long terminal repeat sequence of a human immunodeficiency virus-1 proviral DNA.

3. The protein of claim 1, wherein the target gene comprises a retroviral gene, an adenoviral gene, a foamy viral gene, a parvoviral gene, a foreign gene expressed in a cell, an overexpressed gene, or a misexpressed gene.

4. The protein of claim 1, wherein the chimeric protein comprises a zinc three-finger DNA binding polypeptide linked to a CpG-specific DNA methyltransferase polypeptide.

5. The protein of claim 1, wherein the chimeric protein comprises a mutated Lex A binding polypeptide linked to a cytosine methyltransferase polypeptide.

6. The method of claim 1, wherein the mutated DNA methyltransferase portion comprises at least a portion of a mutated M.SssI DNA methyltransferase protein or at least a portion of a mutated mammalian DNA methyltransferase protein.

7. An expression vector which encodes the chimeric protein of claim 1.

8. The vector of claim 7, wherein the expression vector is replicable.

9. The vector of claim 7, wherein the vector is a pLS vector.

10. The vector of claim 7, wherein the vector is a prokaryotic expression vector, a yeast expression vector, a baculovirus expression vector, a mammalian expression vector, or an episomal mammalian expression vector.

11. A method for inhibiting expression of a target gene which comprises contacting a promoter of the target gene with the chimeric protein of claim 1 so as to specifically methylate the promoter thus inhibiting expression of the target gene.

12. The method of claim 11, wherein the target gene is an endogenous target gene.

13. The method of claim 11, wherein the target gene is a foreign target gene.

14. The method of claim 13, wherein the foreign target gene is a retroviral gene or a viral gene.

15. The method of claim 11, wherein the target gene is associated with a cancer, a central nervous system disorder, a blood disorder, a metabolic disorder, a cardiovascular disorder, an autoimmune disorder, or an inflammatory disorder.

16. The method of claim 15, wherein the cancer is acute lymphocytic leukemia, acute myelogenous leukemia, B-cell lymphoma, lung cancer, breast cancer, ovarian cancer, prostate cancer, lymphoma, Hodgkin's disease, malignant melanoma, neuroblastoma, renal cell carcinoma or squamous cell carcinoma.

17. The method of claim 15, wherein the central nervous system disorder is Alzheimer's disease, Down's syndrome, Parkinson's disease, Huntington's disease, schizophrenia, or multiple sclerosis.

18. The method of claim 15, wherein the infectious disease is cytomegalovirus, herpes simplex virus, human immunodeficiency virus, AIDS, papillomavirus, influenza, candida albicans, mycobacteria, septic shock, or associated with a gram negative bacteria.

19. The method of claim 15, wherein the blood disorder is anemia, hemoglobinopathies, sickle cell anemia, or hemophilia.

20. The method of claim 15, wherein the cardiovascular disorder is familial hypercholesterolemia, atherosclerosis, or renin/angiotensin control disorder.

21. The method of claim 15, wherein the metabolic disorder is ADA, deficient SCID, diabetes, cystic fibrosis, Gaucher's disease, galactosemia, growth hormone deficiency, inherited emphysema, Lesch-Nyhan disease, liver failure, muscular dystrophy, phenylketonuria, or Tay-Sachs disease.

22. The method of claim 15, wherein the autoimmune disorder is arthritis, psoriasis, HIV, or atopic dermatitis.

23. The method of claim 15, wherein the inflammatory disorder is acute pancreatitis, irritable bowel syndrome, Chrone's disease or an allergic disorder.

24. The method of claim 11, wherein the target gene is in a cell.

25. The method of claim 24, wherein the cell is a eukaryotic cell, a bacterial cell, an animal cell, a plant cell, a prokaryotic cell, a virus packaging cell, a somatic cell, a germ cell, a neuronal cell, a myocyte, a T lymphocyte, a CD4⁺ cell, a tumor cell, a CD4⁺ cell, or a stem cell.

26. The method of claim 11, wherein the contacting is by means of liposome mediated delivery, retroviral delivery, gene bombardment, electroporation or cationic precipitation.

27. A method for inhibiting expression of a target gene in a multicellular organism which comprises contacting a promoter sequence of the target gene with the chimeric protein of claim 1, so as to specifically methylate the promoter sequence and thus inhibit expression of the target gene in the multicellular organism.

28. The method of claim 27, wherein the multicellular organism is a plant, an animal or a human.

29. The method of claim 28, wherein the plant is an alfalfa plant, a broccoli plant, a rapeseed plant, a carrot plant, a chicory plant, a coffee plant, a cucurbita plant, a euromelon plant, a potato plant, a raspberry plant, a sunflower plant, a tomato plant, or a wheat plant.

30. The method of claim 28, wherein the animal is a horse, a primate, a porcine animal, a bovine animal, a swine, a fowl, or a fish.

31. The method of claim 27, wherein the chimeric protein or a nucleic acid encoding the chimeric protein is delivered to the multicellular organism via intralesional, intraperitoneal, intramuscular or intravenous injection; liposome-mediated delivery; viral infection; gene bombardment; topical, nasal, oral, anal, ocular or otic delivery.

32. The method of claim 31, wherein the viral infection is via a non-integrating, replication-defective virus.

33. The method of claim 32, wherein the virus comprises a replication-defective Human Immunodeficiency Type 1 provirus, a retroviral vector, an adeno-associated virus, a LNL6 vector, a LXSN vector or a MMuLV retroviral vector.

34. A method of treating a subject infected with a virus which comprises administering to the subject a therapeutic composition comprising the chimeric protein of claim 1, or a nucleic acid molecule encoding the chimeric protein of claim 1, under suitable conditions so as to specifically methylate the viral promoter sequence and inhibit expression of the viral gene thus treating the subject infected with the virus.

35. The method of claim 34, wherein the virus is chosen from the group consisting of a DNA virus, a retrovirus, a herpes virus, an immunodeficiency virus, an adeno-associated virus and an adenovirus.

36. The method of claim 34, wherein the therapeutic composition comprises a nucleic acid molecule encoding a mutated Lex A DNA binding protein portion linked to a mutated DNA methyltransferase protein portion.

37. The method of claim 34, wherein the therapeutic composition comprises a nucleic acid molecule encoding a tridactyl zinc finger DNA binding protein portion capable of specifically binding the Human Immunodeficiency Virus Type 1 5′ long terminal repeat nucleic acid sequence linked to a mutated DNA methyltransferase protein portion.

38. The method of claim 34, wherein the subject is a human.

39. The method of claim 34, wherein the therapeutic composition comprises a replicable expression vector chosen from the group consisting of a pLS vector, a prokaryotic expression vector, a yeast expression vector, a baculovirus expression vector, a mammalian expression vector, and an episomal mammalian expression vector.

40. The method of claim 34, wherein the administration comprises intralesional, intraperitoneal, intramuscular or intravenous injection; liposome-mediated delivery; viral infection; gene bombardment; topical, nasal, oral, anal, ocular or otic delivery.

41. The method of claim 40, wherein the viral infection is via a non-integrating, replication-defective virus.

42. A host cell comprising the expression vector of claim 7.

43. The host cell of claim 42, wherein the host cell is chosen from the group consisting of a eukaryotic cell, a somatic cell, a germ cell, a neuronal cell, a myocyte, a T lymphocyte, a prokaryotic cell, a virus packaging cell, a plant cell, a prokaryotic cell, a tumor cell, a stem cell and a CD4+ cell.

44. A pharmaceutical composition comprising a therapeutically effective amount of the expression vector of claim 7 and a pharmaceutically acceptable carrier.

45. The pharmaceutical composition of claim 44, wherein the carrier comprises a diluent.

46. The pharmaceutical composition of claim 44, wherein the pharmaceutically acceptable carrier is an aerosol, intravenous, oral or topical carrier.

47. A transgenic non-human mammal whose somatic and germ cells contain and express a DNA coding for a chimeric protein of claim 1, the DNA having been stably introduced into the non-human mammal at the single cell stage or an embryonic stage, and wherein the DNA is linked to a promoter and integrated into the genome of the non-human mammal.


SEQUENCE LISTING (8 sequences)

SEQ ID NO: 1 - DNA, 632 bp, HIV
tggaagggctaattcactcccaacgaagacaagatatccttgatctgtggatctaccacacacaaggctacttccctgattagcagaactacacaccagggccaggagtcagatatccactgacctttggatggtgctacaagctagtaccagttgagccagataaggtagaagaggccaacaaaggagagaacaccagcttgttacaccctgtgagcctgcatgggatggatgacccggatatataagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagacctgcatataagcagctgctttttgcctgtactgggtctctctggttagaccagatctgagcctgggagctctctggctagctagggaacccactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 2 - protein, 513 aa, Spiroplasma
  X in this sequence is unknown
MRGSHHHHHHGICTTMSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXGGRSTSHERXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQKDKKAGS

SEQ ID NO: 3 - protein, 20 aa, Spiroplasma
MRGSHHHHHHGICTGTSHER

SEQ ID NO: 4 - protein, 23 aa, Spiroplasma
QKDKKAGSSVDFAIGFGFCFTMS

SEQ ID NO: 5 - protein, 9 aa, Spiroplasma
GGLQPSLIS

SEQ ID NO: 6 - protein, 17 aa, Spiroplasma
MRGSHHHHHHGICTTMS

SEQ ID NO: 7 - protein, 8 aa, Spiroplasma
DKDKKAGS

SEQ ID NO: 8 - DNA, 128 bp, Spiroplasma
ccatggaattgtgagcgctcacaattcgcgtcgcgccgacgccgtcgcggcgacgcgacgcgttggtaccttaacactcgcgagtgttaagcgcagcgcggctgcggcagcgccgctgcgctgcgcaa